(19) United States
(12) Patent Application Publication
(10) Pub. No.: US 2006/0292079 A1
(43) Pub. Date: Dec. 28, 2006

(54) Conjugates of hydroxypyridinone derivative metal complexes with biomolecules and their use for MRI diagnosis

(76) Inventors: Heribert Schmitt-Willich, Berlin (DE); Heiko Schirmer, Berlin (DE); Johannes Platzek, Berlin (DE); Stephane Dumas, Cambridge, MA (US); Vincent Jacques, Somerville, MA (US); Thomas Brumby, Berlin (DE); Detlev Suelzle, Berlin (DE); Bernd Misselwitz, Glienicke (DE)

Correspondence Address: FISH & RICHARDSON P.C., PO BOX 1022, MINNEAPOLIS, MN, 55440-1022, US

(21) Appl. No.: 11/313,263

(22) Filed: Dec. 20, 2005

Related U.S. Application Data
(60) Provisional application No. 60/638,626, filed on Dec. 23, 2004.

(51) Int. Cl. A61K 49/10; C07F 5/00

(52) U.S. Cl. 424/9.361; 424/9.364; 534/16

(57) ABSTRACT

The invention relates to conjugates of hydroxypyridinone derivative metal complexes and to their preparation. The conjugates are suitable as contrast agents in NMR diagnosis. A high relaxivity is achieved and the NMRD maximum is raised through a specific design of the ligands.

### CROSS-REFERENCE TO RELATED APPLICATIONS

This application claims priority under 35 U.S.C. § 119 to U.S. Provisional Application Ser. No. 60/638,626, filed Dec. 23, 2004, incorporated by reference in its entirety herein.

### TECHNICAL FIELD

The invention relates to the subject-matter characterized in the claims, i.e. conjugates of hydroxypyridinone derivative metal complexes with biomolecules. The conjugates are suitable for preparing contrast agents for NMR diagnosis.

### BACKGROUND

Accurate diagnosis is a precondition for targeted and successful therapy. The possibilities in the area of diagnosis in particular have increased very greatly in recent years, with NMR diagnosis, for example, being able to demonstrate virtually every anatomical detail selectively and with great accuracy. However, in many cases, the corresponding structures become visible only through the use of contrast agents. In addition, it is possible to design the contrast agents so that they accumulate selectively in the desired target structures. It is possible thereby to increase the accuracy of imaging while simultaneously reducing the required amount of contrast agent.

Chelate complexes of paramagnetic metals are suitable as contrast agents for NMR diagnosis. The theory and application of gadolinium(III) chelates as NMR contrast agents are explained in detail in a review article by P. Caravan et al. in Chem. Rev. 1999, 99, 2293-2352.

The image intensity in proton NMR is essentially determined by the water protons. It depends on the nuclear relaxation times. Complexes of paramagnetic transition metals and lanthanoids shorten the relaxation times of adjacent protons through dipolar interactions. Paramagnetic contrast agents are not detected directly; on the contrary, there is indirect detection based on the fact that the contrast agents are able to alter relaxation times of adjacent protons such as water protons. Owing to their high magnetic moments and relaxation efficiency, Gd³⁺, Fe³⁺ and Mn²⁺ are preferred paramagnetic metal cations in NMR diagnosis.

An important physical quantity which describes the relaxation behaviour of protons is the longitudinal relaxation time T₁. Tissues with short T₁ relaxation times generally provide images of greater intensity than those with longer relaxation times. Plotting the reciprocal of the measured T₁ relaxation time as a function of the concentration c for a particular paramagnetic compound results in straight lines of slope R. This slope is also called the relaxivity, which is a measure of the ability of the corresponding paramagnetic ion to shorten the relaxation time of the adjacent protons.

Owing to the relatively high toxicity of some of the ions required, ordinarily they are administered not in the form of water-soluble salts but in the form of chelate complexes. The latter can be excreted virtually unchanged from the body. Smaller complexes in solution have a lower moment of inertia and rotate faster in solution (tumbling motion time). A complex which rotates faster has a lower relaxivity. The relaxivity thus increases with the molecular mass of the complete complex. A high molecular mass can be achieved by attachment to macromolecules. A good NMR contrast agent is distinguished inter alia by having a large value for the relaxivity.

Conjugates of Gd-DTPA (diethylenetriaminepentaacetic acid) with albumin are described for example by M. D. Ogan et al. in Invest. Radiol. 1987, 22, 665-671 and U. Schmiedl et al. in Radiology 1987, 162, 205-210. Conjugates of macrocyclic metal complexes and biomolecules are disclosed in WO 95/31444. To improve the selectivity of contrast agents, WO 01/08712 proposes a contrast agent which includes at least two metal chelate units as image-improving groups and at least two “target binding moieties” for binding the contrast agent molecule to the desired target molecule or target organ in the body.

Large contrast agent molecules with high molecular mass are obtained according to WO 97/02051 by incorporating macrocyclic metal complexes in cascade polymers.

Tetraazocyclododecanetetraacetic acid derivatives of high stability and good solubility owing to the lack of charge, which are suitable for attachment to biomolecules, are described in EP-A-0 565 930.

The attachment, described above, of macrocyclic metal complexes to biomolecules makes it possible to increase both the relaxivity and the selectivity of the contrast agent. However, the relaxivities achieved by the immobilization are still so low that the compounds can be employed only poorly as markers for particularly specifically binding biomolecules, because the concentration in the target tissue is so low that the signal provided is too low or at least too noisy. However, detection of a low-noise signal is a precondition for obtaining diagnostically unambiguous information. For this reason there is still a need in NMR diagnosis for metal complexes which produce a significant signal on conjugation with biomolecules even in very low concentrations.

Besides the problem described above, that NMR contrast agents should have a relaxivity which is as high as possible, it is desirable for these contrast agents to have their NMRD maximum in a region which is as suitable as possible for application together with clinical NMR diagnostic instruments. Clinical NMR diagnostic instruments employed at present normally operate at 60 MHz. By contrast, the NMRD maximum of known NMR contrast agents is generally no more than about 20 MHz. There is thus a need for NMR contrast agents with an NMRD maximum which is shifted to high field.

A further ligand class suitable for preparing NMR contrast agents is described by S. M. Cohen et al. in Inorg. Chem. 2000, 39, 5747-5756 and in U.S. Pat. No. 5,624,901. This comprises hydroxypyridinone derivatives, but they are not suitable for attachment to biomolecules.

Hydroxypyridinone and hydroxypyrimidone chelating agents and their gadolinium(III) complexes are also described in WO 03/016923. Some of these compounds exhibit high relaxivities but due to the high molecular weight of the compounds their metal content is rather low. This results in a high absolute dosis needed to be injected compared to the compounds of the invention.

Hydroxypyridinone and hydroxypyrimidone chelating agents and their gadolinium(III) complexes are also described in WO 03/016923. Gadolinium (III) complexes based on hydroxypyridinone and terephthalamide are described by K. N. Raymond in Abstracts of Papers, 227^{th} ACS National Meeting, Anaheim, Calif., United States, Mar. 28-Apr. 1, 2004 (2004) and K. N. Raymond et al. in Abstracts of Papers, 228^{th} ACS National Meeting, Philadelphia, Pa., United States, Aug. 22-26, 2004 (2004). Also in Abstracts of Papers, 227^{th} ACS National Meeting, Anaheim, Calif., United States, Mar. 28-Apr. 1, 2004 (2004) D. G. Churchill at al. describe catecholamide (CAM)-, terephthalamide (TAM)-, hydroxypyridone (HOPO)- and hydroxypyrimidone (HOPY)-based ligand systems for iron sequestration and as gadolinium MRI contrast agents, M. K. Thompson et al. describe lanthanide complexes with tripodal hydroxypyridonate (HOPO)-based ligands, and E. J. Werner et al. describe hydroxypyridinone (HOPO)-based Gd(III) complexes of high stability.

M. K. Thompson in J. Am. Chem. Soc. 2003, 125(47), 14274-5 discloses a heteropodal Gd(III) chelate which is based on a hydroxypyridinate (HOPO)-terephthalamide (TAM) ligand design.

The thermodynamic stability of the Gd(III) complexes of five hexadentate ligands, which incorporate the 2,3-dihydroxyterephthalamide and 2,3-hydroxypyridonate chelating moieties are disclosed by G. Xu et al. in Inorg. Chem. 2004, 43(18), 5492-4.

These chelating agents are suitable for attachment inter alia to biomolecules, the attachment taking place by means of a reactive group in a side chain of the hydroxypyridinone or hydroxypyrimidone chelator. The direct neighbourhood between the coordinating oxygen atoms of the chelating agent and of the reactive group provided for attachment to a biomolecule makes it necessary to attach the chelator to the biomolecule before the complexation with the gadolinium ion, in order to avoid complexation between the gadolinium ion and the reactive group, for example to form particularly stable 5-membered rings. A problem associated with this reaction sequence is that the complexation between the chelator and the gadolinium ion requires drastic reaction conditions which may lead to impairment or even destruction of the previously attached biomolecule. Preparation of conjugates with particularly sensitive biomolecules such as, for example, antibodies is thus not possible, or is possible only with great effort, using the chelators disclosed in WO 03/016923.

### SUMMARY

One object of the present invention is thus to provide contrast agents for NMR diagnosis which solve the problems described above. It is intended in particular that these NMR contrast agents have a relaxivity which is as high as possible, accumulate as selectively as possible at a desired site in the body, and have an NMRD maximum which is particularly suitable for employing the agents together with clinical NMR diagnostic instruments. It is further intended that the NMR contrast agents have good solubility in water, the specificity of the biomolecules should not be impaired by the attachment of the chelators, and the conjugates should be just as well tolerated as the unconjugated biomolecules. Finally, the stability of the conjugates should be as high as possible.

It has now been found that this object can surprisingly be achieved by combining three hydroxypyridinone, hydroxypyrimidone and/or catechol residues by means of a linker in one ligand which is then in turn attached via this linker to a biomolecule. Through the specific design of the ligands, the relaxivity of the resulting contrast agent is increased and, in addition, the NMRD maximum is shifted to higher field by comparison with previously disclosed compounds. Furthermore, attachment of the ligand via the linker instead of via one of the hydroxypyridinone or hydroxypyrimidone residues allows the possibility of preparing the complex with the metal ion to be coordinated before attaching the ligand to the biomolecule without the risk of a side reaction and thus unwanted coordination of the reactive group intended for the reaction with the biomolecule. The finished complex can then be attached under mild reaction conditions even to sensitive biomolecules such as, for example, antibodies.

Accordingly, provided herein are conjugates of the general formula I:
(K)₃—A—U—X′-Bio I,
in which K is independently of one another a radical: 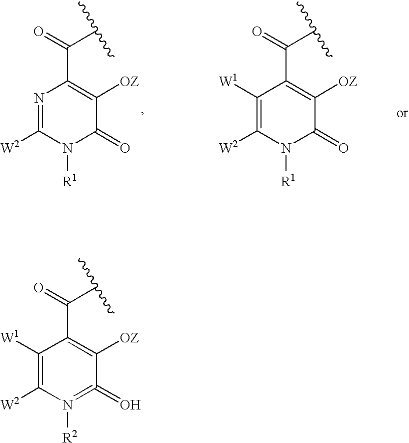
in which Z is a hydrogen atom or a metal ion equivalent, R¹ is a hydrogen atom or a straight-chain or branched, saturated or unsaturated C₁₋₁₀-alkyl radical which is optionally interrupted by 1-3 oxygen atoms, 1-3 nitrogen atoms and/or 1-3 —NR³ radicals, is optionally substituted by 1-4 hydroxy groups, 1-2 carboxyl (optionally present in protected form), 1-2 —SO₃H (optionally present in protected form), 1-2 —PO₃H₂ groups and/or 1-2 halogen atoms, and/or in which optionally 1-2 carbon atoms are present as carbonyl groups, where the alkyl radical or a part of the alkyl radical may be in cyclic form, R² is a hydrogen atom, a straight-chain or branched, saturated or unsaturated C₁₋₁₀-alkyl radical which is optionally interrupted by 1-3 oxygen atoms, 1-3 nitrogen atoms and/or 1-3 —NR³ radicals, is optionally substituted by 1-2 hydroxy groups, 1-2 carboxyl, 1-2 —SO₃H, 1-2 —PO₃H₂ groups and/or 1-2 halogen atoms, and/or in which optionally 1-2 carbon atoms are present as carbonyl groups, where the alkyl radical or a part of the alkyl radical may be in cyclic form, —COOH—, halogen, —CONR³R⁴, —SO₃H or —PO₃H₂,

R³ and R⁴ are independently of one another a hydrogen atom or a straight-chain, branched or cyclic, saturated or unsaturated C₁₋₁₀-alkyl radical which is optionally substituted by 1-4 hydroxy groups or interrupted by 1-2 oxygen atoms,

W¹ and W² are independently of one another a radical R¹ or —CONR³R⁴,

A is a radical: 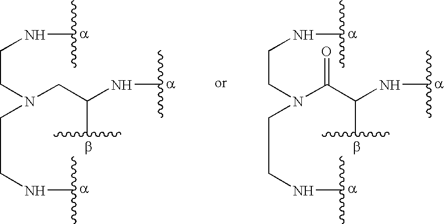
in which the positions α are linked to K and the positions β are linked to U,

U is a direct linkage or a straight-chain or branched, saturated or unsaturated C₁₋₂₀-alkylene radical which is optionally interrupted by 1-4 oxygen atoms, 1-4 sulphur atoms, 1-4 nitrogen atoms, 1-4 —NR³ radicals, 1-4 —NHCO radicals, 1-4 —CONH radicals, 1-4 —O—P(═O) (OH)—O— radicals and/or 1-2 arylene radicals, is optionally substituted by 1-3 straight-chain, branched or cyclic, saturated or unsaturated C₁₋₁₀-alkyl radicals, 1-3 hydroxy groups, 1-3 carboxyl groups, 1-3 aryl groups, 1-3 halogen atoms and/or 1-3 —O—C₁₋₆-alkyl groups (where the alkyl radical is straight-chain, branched or cyclic, saturated or unsaturated), and/or in which optionally 1-3 carbon atoms may be present as carbonyl groups, where the alkylene radical or a part of the alkylene radical may be in cyclic form, and

X is a group able to enter into a reaction with a biomolecule, and

Bio is the residue of a biomolecule and the salts thereof.

U can be selected from the group consisting of —CH₂—CH₂—, —CH₂—CH₂—CO—NH—CH₂—CH₂—, —CH₂—CO—NH—CH₂—, —CH (CH₃) —CO—NH—CH₂—CO—NH—CH₂—CH₂—, —CH₂-phenylene-, -phenylene-, -cyclohexylene-, —CH₂-phenylene-O—CH₂—, —CH₂-phenylene-O—CH₂—CO—NH—CH₂—CH₂—, -phenylene-O—CH₂—, —CO-phenylene-, —CO-phenylene-CO—NH—CH₂—CH₂—, —(CH₂)₄—, —(CH₂)₄—NH—CO—CH₂—CH₂— and —(CH₂)₄—NH—CO—CH₂—O—CH₂—, where these radicals are linked in the direction of reading on the left to A and in the direction of reading on the right to X.

X′ is a radical of a group X and X can be selected from the group consisting of carboxyl, activated carboxyl, amino, isocyanate, isothiocyanate, hydrazine, semicarbazide, thiosemicarbazide, chloroacetamide, bromoacetamide, iodoacetamide, acylamino, mixed anhydrides, azide, hydroxide, sulphonyl chloride, carbodiimide, pyridyl-CH═CH₂ and radicals of the formulae: 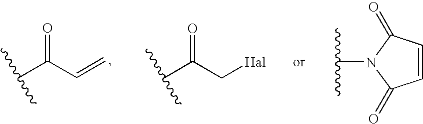
in which Hal is a halogen atom.

An activated carboxyl group can be selected from:

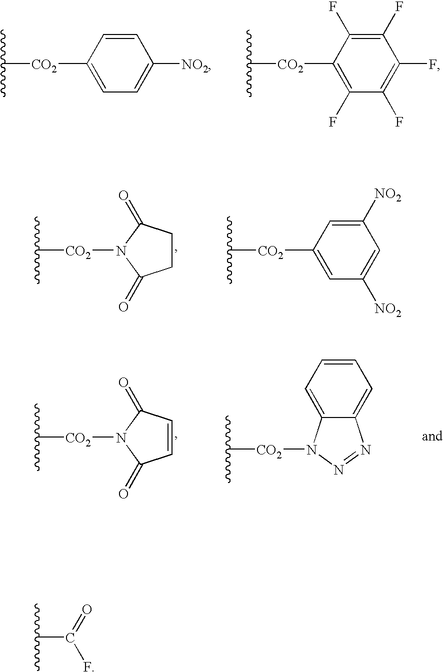

A biomolecule can be selected from the group consisting of biopolymers, proteins, synthetically modified biopolymers, carbohydrates, antibodies, DNA and RNA fragments, β-amino acids, vector amines for importation into the cell, biogenic amines, pharmaceuticals, oncological preparations, synthetic polymers directed at a biological target, steroids, prostaglandins, Taxol and its derivatives, endothelins, alkaloids, folic acid and its derivatives, bioactive lipids, fats, fatty acid esters, synthetically modified mono-, di- and triglycerides, liposomes which are derivatized on the surface, micelles of natural fatty acids or of perfluoroalkyl compounds, porphyrins, texaphrins, extended porphyrins, cytochromes, inhibitors, neuraminidases, neuropeptides, immunomodulators, endoglycosidases, substrates which are attacked by the enzymes, calmodulin kinase, casein kinase II, glutathione S-transferase, heparinase, matrix metalloproteases, β-insulin receptor kinase, UDP-galactose 4-epimerase, fucosidases, G-proteins, galactosidases, glycosidases, glycosyl transferases and xylosidase, antibiotics, vitamins and vitamin analogues, hormones, DNA intercalators, nucleosides, nucleotides, lectins, vitamin B12, Lewis-X and related substances, psoralens, diene/triene antibiotics, carbacyclins, VEGF, somatostatin and its derivatives, biotin derivatives, antihormones, tumour-specific proteins and synthetics, polymers which accumulate in acidic or basic regions of the body, myoglobins, apomyoglobins, neurotransmitter peptides, tumour necrosis factors, peptides which accumulate in inflamed tissues, blood pool reagents, anions and cation transporter proteins, polyesters, polyamides and polyphosphates.

A conjugate can hve at least two of the radicals Z be a metal ion equivalent of a paramagnetic element of atomic numbers 21-29, 42, 44 or 58-70.

Also provided herein is a process for preparing conjugates of the general formula I:
(K)₃—A—U—X′-Bio I,
in which K, A, U, X′ and Bio are as defined previously, in which a compound of the general formula II:
(K)₃—A—U—X II,
in which K, A and U are as defined above and X is a group which is able to enter into a reaction with a biomolecule, is reacted with a biomolecule and if desired is subsequently reacted in a manner known per se with at least one metal oxide or metal salt of a desired element, and where appropriate subsequently acidic hydrogen atoms still present in the complexes obtained in this way are replaced wholly or partly by cations of inorganic and/or organic bases, amino acids or amino amides.

Also disclosed herein are pharmaceutical compositions comprising at least one physiologically acceptable conjugate provided herein, if desired with the additives customary in pharmaceuticals.

Also disclosed herein is the use of a conjugate for preparing agents for NMR diagnosis.

Also provided is a process for preparing a conjugate of the general formula I:
(K)₃—A—U—X′-Bio I,
in which K, A, U, X′ and Bio are as defined previously, in which a compound of the general formula II:
(K)₃—A—U—X II,
in which K, A and U are as defined above and X is a group which is able to enter into a reaction with a biomolecule, is reacted with a biomolecule to form a complex.

The process can further comprise reacting the complex with at least one metal oxide or metal salt of a desired element.

A process can also further include replacing wholly or partly one or more acidic hydrogen atoms still present in the complex with one or more cations of inorganic and/or organic bases, amino acids or amino amides.

### DETAILED DESCRIPTION

The present invention thus relates to conjugates of the general formula I:
(K)₃—A—U—X′-Bio I,
in which K is independently of one another a radical 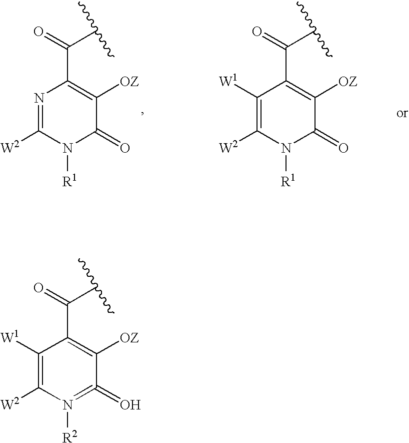
in which Z is a hydrogen atom or a metal ion equivalent, R¹ is a hydrogen atom or a straight-chain or branched, saturated or unsaturated C₁₋₁₀-alkyl radical which is optionally interrupted by 1-3 oxygen atoms, 1-3 nitrogen atoms and/or 1-3 —NR³ radicals, is optionally substituted by 1-4 hydroxy groups, 1-2 carboxyl (optionally present in protected form), 1-2 —SO₃H (optionally present in protected form), 1-2 —PO₃H₂ groups and/or 1-2 halogen atoms, and/or in which optionally 1-2 carbon atoms are present as carbonyl groups, where the alkyl radical or a part of the alkyl radical may be in cyclic form, R² is a hydrogen atom, a straight-chain or branched, saturated or unsaturated C₁₋₁₀-alkyl radical which is optionally interrupted by 1-3 oxygen atoms, 1-3 nitrogen atoms and/or 1-3 —NR³ radicals, is optionally substituted by 1-2 hydroxy groups, 1-2 carboxyl, 1-2 —SO₃H, 1-2 —PO₃H₂ groups and/or 1-2 halogen atoms, and/or in which optionally 1-2 carbon atoms are present as carbonyl groups, where the alkyl radical or a part of the alkyl radical may be in cyclic form, —COOH, halogen, —CONR³R⁴, —SO₃H or —PO₃H₂,

R³ and R⁴ are independently of one another a hydrogen atom or a straight-chain, branched or cyclic, saturated or unsaturated C₁₋₁₀-alkyl radical which is optionally substituted by 1-4 hydroxy groups or interrupted by 1-2 oxygen atoms,

W¹ and W² are independently of one another a radical R¹ or —CONR³R⁴,

A is a radical: 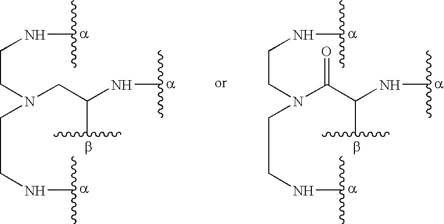
in which the positions α are linked to K and the positions β are linked to U,

U is a direct linkage or a straight-chain or branched, saturated or unsaturated C₁₋₂₀-alkylene radical which is optionally interrupted by 1-4 oxygen atoms, 1-4 sulphur atoms, 1-4 nitrogen atoms, 1-4 —NR³ radicals, 1-4 —NHCO radicals, 1-4 —CONH radicals, 1-4 —O—P(═O) (—OH)—O— radicals and/or 1-2 arylene radicals, is optionally substituted by 1-3 straight-chain, branched or cyclic, saturated or unsaturated C₁₋₁₀-alkyl radicals, 1-3 hydroxy groups, 1-3 carboxyl groups, 1-3 aryl groups, 1-3 halogen atoms and/or 1-3 —O—C₁₋₆ alkyl groups (where the alkyl radical is straight-chain, branched or cyclic, saturated or unsaturated), and/or in which optionally 1-3 carbon atoms are present as carbonyl groups, where the alkylene radical or a part of the alkylene radical may be in cyclic form, and

X′ is the radical of a group X which has entered into a reaction with a biomolecule, and Bio is the radical of a biomolecule, and salts thereof and to the use thereof for preparing agents for NMR diagnosis.

Unless indicated otherwise, in the present text “alkyl radical” or “alkylene radical” mean a saturated or unsaturated, straight-chain or branched or cyclic alkyl(ene) radical having the indicated number of carbon atoms. If this radical may contain further groups or atoms or may be interrupted thereby, this means that the further groups or atoms are present in addition to the atoms already present in the radical and can be inserted at any position on the radical, including the terminal positions.

“Aryl” preferably means phenyl, biphenylyl, pyridyl, furanyl, pyrrolyl and imidazolyl. Phenyl and pyridyl are particularly preferred.

The inventive conjugates of the formula I include three hydroxypyridinone, hydroxypyrimidone and/or catechol residues. These residues contribute to the coordination and to balancing the charge of a coordinated metal ion. Z is therefore either a hydrogen atom or a metal ion equivalent.

The hydroxypyridinone or hydroxypyrimidone residue which may represent K in the general formula I carries a substituent R¹ which is a hydrogen atom or a straight-chain or branched, saturated or unsaturated C₁₋₁₀-alkyl radical which is optionally interrupted by 1-3 oxygen atoms, 1-3 nitrogen atoms and/or 1-3 —NR³ radicals, is optionally substituted by 1-4 hydroxy groups, 1-2 carboxyl (optionally present in protected form)., 1-2 —SO₃H (optionally present in protected form), 1-2 —PO₃H₂ groups and/or 1-2 halogen atoms and/or in which optionally 1-2 carbon atoms are present as carbonyl groups, where the alkyl radical or a part of the alkyl radical may be in cyclic form.

R¹ is preferably a hydrogen atom or a straight-chain or branched, preferably straight-chain C₁₋₅-alkyl radical which may be interrupted by 1-2 oxygen atoms and/or substituted by 1-4 hydroxy groups, a carboxyl group and/or a group —SO₃H. Preferred examples of R¹ are —H, —CH₃, —CH₂—CH₃, —CH₂—CH₂—CH₃, —CH(CH₃)—CH₃, —C(CH₃) (CH₃)—CH₃, —CH₂—OH, —CH₂—CH₂—OH, —CH₂—CH₂—O—CH₃, —CH₂—COOH, —CH₂—COOt— But, —CH₂—COOCH₂C₆H₅, —CH₂—CH₂—SO₃H, —CH₂—CH₂—CH₂—SO₃H, —CH₂—CH₂—CH₂—CH₂—SO₃H, —CH₂—CH(OH)—CH₂—OH, —CH₂—CH₂—O—CH₂CH₂—O—CH₃, —CH₂—CH₂—O—CH₂—CH₂—OH, —CH₂—CH₂—O—CH₂—COOH and —CH[CH₂—O—CH—(CH₂—OH)₂]₂. Particular preference is given to —H, methoxyethyl, methyl and —CH₂—COOH, especially methoxyethyl and methyl.

W¹ and W² are independently of one another a radical R¹, where R¹ is as defined above and also includes the above preferred radicals. W¹ and W² particularly preferably are independently a hydrogen atom or a straight-chain or branched, preferably straight-chain C₁₋₅-alkyl radical, in particular a hydrogen atom or a methyl radical. It is possible for example for one of W¹ and W² to be a hydrogen atom and the other of W¹ and W² to be a methyl radical, or W¹ and W² may both be a hydrogen atom.

The catechol residue which may alternatively represent K in the formula I carries a substituent R². The latter may be a hydrogen atom, a straight-chain or branched, saturated or unsaturated C₁₋₁₀-alkyl radical which is optionally interrupted by 1-3 oxygen atoms, 1-3 nitrogen atoms and/or 1-3 —NR³ radicals, is optionally substituted by 1-2 hydroxy groups, 1-2 carboxyl, 1-2 —SO₃H, 1-2 —PO₃H₂ groups and/or 1-2 halogen atoms, and/or in which optionally 1-2 carbon atoms are present as carbonyl groups, where the alkyl radical or a part of the alkyl radical may be in cyclic form, —COOH, halogen, —CONR³R⁴, —SO₃H or —PO₃H₂. Preferred alkyl radicals and substituted and heteroatom-interrupted alkyl radicals for R² are those as described above for R¹. Fluorine, chlorine, bromine and iodine are suitable as halogen.

The radicals R³ and R⁴ above are independently of one another a hydrogen atom or a straight-chain, branched or cyclic, saturated or unsaturated C₁₋₆-alkyl radical which is optionally substituted by 1-2 hydroxy groups. Particularly suitable C₁₋₆-alkyl radicals for R³ and R⁴ are methyl, ethyl, propyl, isopropyl, n-butyl, isobutyl, tert-butyl, pentyl, hexyl, cyclohexyl, 2-hydroxyethyl and —CH[CH₂—O—CH—(CH₂—OH)₂]₂.

The three hydroxypyridinone, hydroxypyrimidone and/or catechol residues of the conjugates of the general formula I serve to coordinate a metal ion. In order that the complexes formed have maximum stability, the three complex-forming radicals K of the inventive conjugate are held together by a central tetravalent radical A (linker), so that a multidentate ligand is provided. In addition, this central radical A links not only the three coordinating radicals K together but additionally the latter to a group X′ which has entered into a reaction with a biomolecule.

A in the conjugates of the general formula I is a radical: 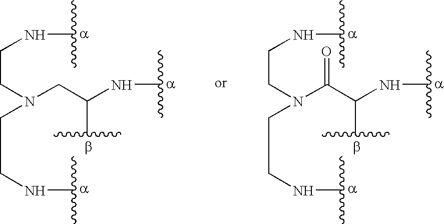
in which the positions α are linked to K and the positions β are linked to U.

The radical (K)₃—A— of the conjugates of the invention has been attached by means of a group X which is able to enter into a reaction with a biomolecule, via a spacer U, to a biomolecule.

The spacer U is in this connection a direct linkage or a straight-chain or branched, saturated or unsaturated C₁₋₂₀-alkylene radical which is optionally interrupted by 1-4 oxygen atoms, 1-4 sulphur atoms, 1-4 nitrogen atoms, 1-4 —NR³ radicals, 1-4 —NHCO radicals, 1-4 —CONH radicals, 1-4 —O—P(═O) (—OH)—O— radicals and/or 1-2 arylene radicals, is optionally substituted by 1-3 straight-chain, branched or cyclic, saturated or unsaturated C₁₋₁₀-alkyl radicals, 1-3 hydroxy groups, 1-3 carboxyl groups, 1-3 aryl groups, 1-3 halogen atoms and/or 1-3 —O—C₁₋₁₆-alkyl groups (where the alkyl radical is straight-chain, branched or cyclic, saturated or unsaturated), and/or in which optionally 1-3 carbon atoms are present as carbonyl groups, where the alkylene radical or a part of the alkylene radical may be in cyclic form. R³ herein is as defined above. Preferred alkyl and aryl groups are likewise as defined above.

In one embodiment of the present invention, U is a phenylene or cyclohexylene radical or a straight-chain or branched, saturated C₁₋₁₀-alkyl radical which may be interrupted by an oxygen atom, an —NR³ radical, one or two amide residue(s) and/or a phenylene radical, and in which one or two carbon atom(s) may be present as carbonyl group(s).

U may be selected for example from the group consisting of —CH₂—CH₂—, —CH₂—CH₂—CO—NH—CH₂—CH₂—, —CH₂—CO—NH—CH₂—, —CH(CH₃)—CO—NH—CH₂—CO—NH—CH₂—CH₂—, —CH₂-phenylene-, -phenylene-, -cyclohexylene-, —CH₂-phenylene-O—CH₂—, —CH₂-phenylene-O—CH₂—CO—NH—CH₂—CH₂—, -phenylene-O—CH₂—, —CO-phenylene-, —CO-phenylene-CO—NH—CH₂—CH₂—, —(CH₂)₄—, —(CH₂)₄—NH—CO—CH₂—CH₂— and —(CH₂)₄—NH—CO—CH₂—O—CH₂—, where these radicals are linked in the direction of reading on the left to A and in the direction of reading on the right to X.

A group X′ is attached via the spacer U to the radical A of the formula I. This group X′ is the radical of a group X which has entered into a reaction with a biomolecule. Suitable examples of X are carboxyl (—COOH), activated carboxyl, amino (—NH₂), isocyanate (—NCO), isothiocyanate (—NCS), hydrazine (—NHNH₂), semicarbazide (—NHCONHNH₂), thiosemicarbazide (—NHCSNHNH₂), chloroacetamide (—NHCOCH₂Cl), bromoacetamide (—NHCOCH₂Br), iodoacetamide (—NHCOCH₂I), acylamino such as, for example, acetylamino (—NHCOCH₃), mixed anhydrides, azide, hydroxide, sulphonyl chloride, carbodiimide, pyridyl-CH═CH₂ or a group of the formulae: 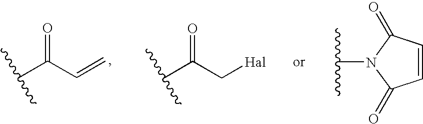
in which Hal is a halogen atom.

Activated carboxyl group above means carboxyl groups derivatized in such a way that they facilitate reaction with a biomolecule. Groups which can be used for the activation are known, and reference may be made for example to M. and A. Bodansky, “The Practice of Peptide Synthesis”, Springerverlag 1984. Examples are adducts of the carboxylic acid with carbodiimides or activated esters such as, for example, hydroxybenzotriazole esters. The activated carboxyl group for X is particularly preferably selected from:

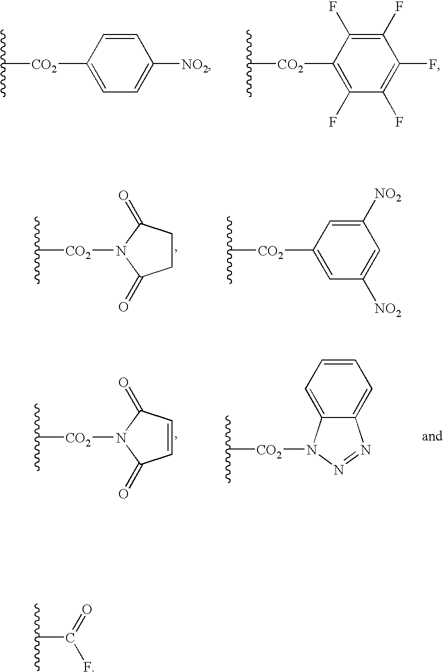

Z in formula I is a hydrogen atom or a metal ion equivalent. Which metal ion is to be present complexed in the inventive compound depends on the intended use of the conjugates with a biomolecule which are prepared with the inventive compounds. Corresponding conjugates are suitable for example for NMR diagnosis. The conjugates are particularly preferably employed as contrast agents in NMR diagnosis.

The preparation of complexes for NMR diagnosis can take place in the way which has been disclosed in the patents EP 71564, EP 130934 and DE-A 34 01 052. This is done by dissolving or suspending the metal oxide or a metal salt (for example a chloride, nitride, acetate, carbonate or sulphate) of the desired element in water, a lower alcohol (such as methanol, ethanol or isopropanol) and/or another organic solvent such as THF, pyridine, etc., and reacting with the solution or suspension of the equivalent amount of the inventive complexing agent. A specific preparation example is disclosed in Inorganic. Chem. 2000, 39, 2652-2660. In this example, the complexing agent is dissolved in methanol/THF, and a solution of the metal salt in methanol/THF is added dropwise to the complexing agent solution. Subsequently, pyridine is added, and the mixture is heated under reflux. The precipitated complex is removed by centrifugation and washed with methanol/THF.

The inventive conjugates are used for NMR diagnosis in the form of their complexes with the ions of the paramagnetic elements having atomic numbers 21-29, 42, 44 and 58-70. Examples of suitable ions are the chromium(III), iron(II), cobalt(II), nickel(II), copper(II), praseodymium(III), neodymium(III), samarium(III) and ytterbium(III) ions. Because of their strong magnetic moment, the gadolinium(III), manganese(II) and iron(III) ions are particularly preferred for NMR diagnosis.

The inventive compounds and especially their conjugates with biomolecules satisfy the diverse requirements for suitability as contrast agents for magnetic resonance imaging. Thus, they are outstandingly suitable for improving the information provided by the image obtained with the aid of magnetic resonance imaging, through increasing the signal intensity, after oral or parenteral administration. In addition, they show high activity, which is necessary in order to expose the body to minimum amounts of foreign substances, and the good tolerability which is necessary in order to maintain the non-invasive character of the investigations.

It is additionally possible for the inventive complex compounds advantageously to be used as susceptibility reagents and as shift reagents for in vivo NMR spectroscopy.

Neutralization of any free carboxyl groups which are still present takes place with the aid of inorganic bases (e.g. hydroxides, carbonates or bicarbonates) of, for example, sodium, potassium, lithium, magnesium or calcium and/or organic bases such as, inter alia, primary, secondary and tertiary amines such as, for example, ethanolamine, morpholine, pyridine, glucamine, N-methyl- and N,N-dimethylglucamine, and basic amino acids such as, for example, lysine, arginine and ornithine or of amides of originally neutral or acidic amino acids.

The neutral complex compounds can be prepared for example by adding sufficient desired base to acidic complex salts in aqueous solution or suspension until the neutral point is reached. The resulting solution can then be concentrated to dryness in vacuo. It is frequently advantageous to precipitate the neutral salts which have formed by adding water-miscible solvents such as, for example, lower alcohols (methanol, ethanol, isopropanol and others), lower ketones (acetone and others) and/or polar ethers (tetrahydrofuran (THF), dioxane, 1,2-dimethoxyethane and others) and thus obtain crystals which are easily isolated and readily purified. It has proved particularly advantageous to add the desired base to the reaction mixture even during the complexation and thus save one step of the process. A particularly advantageous preparation process using methanol/THF as solvent and pyridine as base is described in Inorg. Chem. 2000, 39, 2652-2660.

The inventive conjugates of the formula I can be prepared by processes known to the skilled person. For example, the conjugates of the formula I can be obtained by a process in which a compound of the formula II:
(K)₃—A—U—X II,
in which K, A and U are as defined above, and X is a group able to enter into a reaction with a biomolecule, is reacted with a biomolecule, and subsequently if desired is reacted in a manner known per se with at least one metal oxide or metal salt of a desired element, and where appropriate subsequently acidic hydrogen atoms still present in the complexes obtained in this way are replaced wholly or partly by cations of inorganic and/or organic bases, amino acids or amino amides.

The compounds of the formula II can be prepared by processes known to the skilled person. For example, the compounds of the formula II can be obtained by a process in which a compound of the formula III:
A′—U—X III,
in which U and X are as defined above, and A′ is the precursor to the tetravalent radical A, is reacted with Nu-K, where K is as defined above, K and X are optionally in their protected form, and Nu is a nucleofuge, subsequently the protective groups which are present where appropriate are removed, and if desired is reacted in a manner known per se with at least one metal oxide or metal salt of a desired element, and where appropriate subsequently acidic hydrogen atoms still present in the complexes obtained in this way are replaced wholly or partly by cations of inorganic and/or organic bases, amino acids or amino amides.

Radicals used as nucleofuge are, for example:

Cl, Br, I, O-triflate, mesylate and tosylate.

The reaction is carried out for example in a mixture of water and organic solvents such as: isopropanol, ethanol, methanol, butanol, dioxane, tetrahydrofuran, dimethylformamide, dimethylacetamide, formamide or dichloromethane. Ternary mixtures of water, isopropanol and dichloromethane are preferred.

The reaction is carried out in a temperature range between −10° C. and 100° C., preferably between 0° C. and 30° C.

Numerous possibilities are known to the skilled person for protecting the groups named above. The embodiments described below serve to illustrate these protective group techniques without being restricted to these synthetic routes.

Suitable acid protective groups are C₁₋₆-alkyl, C₆₋₁₀-aryl and C₆₋₁₀-ar(C₁₋₄)-alkyl groups, and trialkylsilyl groups. Preference is given to the methyl, ethyl, propyl, isopropyl, n-butyl, i-butyl and tert-butyl groups.

These acid protective groups are eliminated by processes known to the skilled person, for example by hydrolysis, hydrogenolysis, alkaline hydrolysis of the esters with alkali in hydroalcoholic solution at temperatures from 0 to 50° C., acidic hydrolysis with mineral acids or, in the case of tert-butyl esters, with the aid of trifluoroacetic acid.

The NH groups can be protected and liberated again in diverse ways. The N-trifluoroacetyl derivative is cleaved by potassium carbonate or sodium carbonate in water (H. Newman, J. Org. Chem., 30: 287 (1965), M. A. Schwartz et al., J. Am. Chem. Soc., 95 G12 (1973) ) or simply by ammonia solution (M. Imazama and F. Eckstein, J. Org. Chem., 44: 2039 (1979)). The tert-butyloxycarbonyl derivative can likewise be cleaved under mild conditions: it is sufficient to stir with trifluoroacetic acid (B. F. Lundt et al., J. Org. Chem., 43: 2285 (1978)). The group of NH protective groups which can be cleaved by hydrogenolysis or reduction is very large: the N-benzyl group can conveniently be cleaved with hydrogen/Pd-C (W. H. Hartung and R. Rimonoff, Org. Reactions VII, 262 (1953)), which also applies to the trityl group (L. Zervas et al., J. Am. Chem. Soc., 78: 1359 (1956)) and the benzyloxycarbonyl group (M. Bergmann and L. Zervas Ber. 65: 1192 (1932)).

Activated esters of the compounds described above are prepared as known to the skilled person. In the case of isothiocyanates or α-haloacetates, the corresponding terminal amine precursors are reacted by methods known from the literature with thiophosgene or 2-haloacetyl halides. Reaction with appropriately derivatized esters of N-hydroxysuccinimide such as, for example: 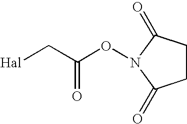
is also possible (Hal=halogen).

It is generally possible to use for this purpose all customary methods for activating carboxylic acids which are known in the state of the art. The molecule Nu-K is preferably first synthesized independently. If the molecule contains an amide group, this is prepared for example by reacting an activated carboxylic acid with an amine. The carboxylic acid is activated by customary methods. Examples of suitable activating reagents are dicyclohexylcarbodiimide (DCC), 1-ethyl-3-(3-dimethyl-aminopropyl)carbodiimide hydrochloride (EDC), benzo-triazol-1-yloxytris(dimethylamino)phosphonium hexafluoro-phosphate (BOP) and O-(benzotriazol-1-yl)-1,1,3,3-tetramethyluronium hexafluorophosphate (HBTU), preferably DCC. It is also possible to add O-nucleophilic catalysts such as, for example, N-hydroxysuccinimide (NHS) or N-hydroxybenzotriazole.

If the group X is a carboxylic acid function, this can be employed in protected form (e.g. in the form of the benzyl ester), and elimination of the protective group can then take place by hydrogenolysis.

In order to link this carboxylic acid function to a suitable functional group of a suitable biomolecule, the latter should ordinarily first be activated. This is preferably done by intermediate generation of activated esters which are then attacked by a nucleophilic group of the biomolecule. This results in a covalent linkage between the biomolecule and the inventive compound of the formula I. Preferred activated esters are the esters of N-hydroxysuccinimide, the esters of paranitrophenol or the esters of pentafluorophenol. If the group X is to linked in the form of an isothiocyanate to the biomolecule there is preferably initial use of a terminal amine which can, if necessary, be provided with a suitable protective group. Suitable protective groups are known from peptide chemistry. After elimination of the protective group, the isothiocyanate can be generated by reacting the primary terminal amine with thiophosgene. Nucleophilic groups of the biomolecule can be added onto this isothiocyanate.

In one embodiment, the group X is a maleimide which can, for example, react selectively with thiol functions of the biomolecule.

In another embodiment, the group X is a nucleophile (NH₂, SH) which attacks a suitable functionality of the biomolecule (activated ester, maleimide, etc.). Numerous biomolecules functionalized with maleimides are commercially available.

The conjugates are ordinarily synthesized by firstly generating a derivatized and functionalized chelate complex, which is then linked to the biomolecule. However, it is also possible in the case of synthetically prepared biomolecules to incorporate the inventive chelate complex into the biomolecule during the synthesis thereof. This can take place for example during the sequential synthesis of oligopeptides in a robotic synthesizer. If necessary, for this purpose the protective groups which are customary in the synthesis of the appropriate biomolecule can be introduced into the inventive compound. They are then eliminated again during the course of the customary synthesis algorithms on the synthesizer.

“Biomolecule” in the present case means every molecule which either occurs naturally for example in the body or has been prepared synthetically with an analogous structure. It also means molecules able to interact with a molecule occurring biologically, for example in the body, or with a structure occurring therein, so that for example the conjugates accumulate at certain desired sites in the body. “Body” means in the present case any plant or animal body, with preference for animal and especially human bodies.

Biomolecules are in particular the molecules which occur in organisms and which as products of evolutionary selection fulfil specific tasks through ordered and complex interaction for the organism and comprise the basis for its vital functions (metabolism and metamorphosis, reproduction, energy balance). In biomolecules, simple building blocks (amino acids, nucleobases, monosaccharides, fatty acids, etc.) are usually assembled into larger molecules (proteins, nucleic acids, polysaccharides, lipids, etc.). Corresponding macromolecules are also referred to as biopolymers.

It is possible and advantageous for the biomolecule to have for example a polypeptide structure composed of amino acids with side chains able to enter into a reaction with the reactive group X of the inventive compounds of the formula I. Such side chains include for example the carboxyl groups of aspartic acid and glutamic acid residues, the amino groups of lysine residues, the aromatic groups of tyrosine and histidine residues and the sulphhydryl groups of cysteine residues.

A review of biomolecules with numerous examples is to be found in the lecture notes “Chemie der Biomoleküle” of the Technical University of Graz (H. Berthold et al., Institut für Organische Chemie, TU-Graz, 2001), which can also be viewed via the internet under www.orgc.tu-graz.ac.at. The contents of this document are included by reference in the present description.

The following biomolecules are particularly suitable for forming conjugates with the inventive compounds:

Biopolymers, proteins such as proteins which have biological function, HSA, BSA, etc., proteins and peptides which accumulate at particular sites in the organism (e.g. at receptors, cell membranes, channels etc.), peptides which can be cleaved by proteases, peptides having synthetic intended breakage sites (e.g. labile esters, amides etc.), peptides which are cleaved by metalloproteases, peptides having photocleavable linkers, peptides having groups cleavable by oxidative agents (oxidases), peptides having natural and unnatural amino acids, glycoproteins (glycopeptides), signal proteins, antiviral proteins and apoctosis, synthetically modified biopolymers, such as biopolymers derivatized with linkers, modified metalloproteases and derivatized oxidase etc., carbohydrates (mono- to polysaccharides) such as derivatized sugars, sugars which can be cleaved in the organism, cyclodextrins and its derivatives, aminosaccharides, chitosan, polysulphates and acetylneuramininc acid derivatives, antibodies such as monoclonal antibodies, antibody fragments, polyclonal antibodies, minibodies, single chains (also those linked to linkers to give multiple fragments), red blood corpuscles and other constituents of blood, cancer markers (e.g. CAA) and cell adhesion substances (e.g. Lewis X and anti-Lewis X derivatives), DNA and RNA fragments such as derivatized DNAs and RNAs (e.g. those found by the SELEX method), synthetic RNA and DNA (also with unnatural bases), PNAs (Hoechst) and antisense β-amino acids (Seebach), vector amines for importation into the cell, biogenic amines, pharmaceuticals, oncological preparations, synthetic polymers which are directed at a biological target (e.g. receptor), steroids (natural and modified), prostaglandins, Taxol and its derivatives, endothelins, alkaloids, folic acid and its derivatives, bioactive lipids, fats, fatty acid esters, synthetically modified mono-, di- and triglycerides, liposomes which are derivatized on the surface, micelles from natural fatty acids or from perfluoroalkyl compounds, porphyrins, texaphrins, extended porphyrins, cytochromes, inhibitors, neuraminidases, neuropeptides, immunomodulators such as FK 506, CAPE and gliotoxin, endoglycosidases, substrates which are activated by enzymes such as calmodolin kinase, casein kinase II, glutathione S-transferase, heparinase, matrix metalloproteases, β-insulin receptor kinase, UDP-galactose 4-epimerase, fucosidases, G-proteins, galactosidases, glycosidases, glycosyltransferases and xylosidase, antibiotics, vitamin and vitamin analogues, hormones, DNA intercalators, nucleosides, nucleotides, lectins, vitamin B12, Lewis X and related substances, psoralens, diene/triene antibiotics, carbacyclins, VEGF (vascular endothelial growth factor), somatostatin and its derivatives, biotin derivatives, antihormones, tumour-specific proteins and synthetics, polymers which accumulate in acidic or basic regions of the body (pH-control distribution), myoglobins, apomyoglobins etc., neurotransmitter peptides, tumour necrosis factors, peptides which accumulate in inflamed tissue, blood pool reagents, anions and cation transporter proteins, polyesters (e.g. of lactic acid), polyamides and polyphosphates.

Most of the aforementioned biomolecules are commercially available, for example from Merck, Aldrich, Sigma, Calbiochem or Bachem.

It is additionally possible to employ as biomolecules all the “plasma protein binding groups” and “target binding groups” disclosed respectively in WO 96/23526 and in WO 01/08712. The contents of these two publications are therefore included by reference in the present description.

In principle there can be any number of compounds of the formula II per biomolecule, but a molecular ratio of from 0.1:1 to 10:1 is preferred, in particular from 0.5:1 to 2:1.

The compounds of the formula II are also suitable for conjugation with all molecules which are reacted in the state of the art with fluorescent dyes in order, for example, to determine their location inside the cell by epifluorescence microscopy. The compounds can also be conjugated with in principle any medicaments in order then to follow transport within the organism after administration of the medicament by the NMR technique. It is also possible for the inventive conjugates to contain further additional molecules which have been conjugated to the biomolecules. The term “biomolecule” thus includes for the purposes of the invention all molecules which occur in biological systems and all molecules which are biocompatible.

The inventive conjugates are preferably employed as contrast agents in NMR diagnosis. The conjugates should therefore be soluble in water. If the conjugates obtained with the inventive compounds are to be employed as NMR contrast agents, the dose thereof is preferably an amount of 0.0001-5 mmol/kg of bodyweight and particularly preferably an amount of 0.005-0.5 mmol/kg of bodyweight. Details of use are discussed for example in H.-J. Weinmann et al., Am. J. of Roentgenology 142, 619 (1984). The surprisingly high relaxivity of the inventive conjugates with simultaneous target specificity means that particularly low doses thereof can be used for example to detect tumours.

The inventive compounds are particularly distinguished by having the highest relaxivities described to date for metal complex conjugates in the immobilized state. This is particularly important because the conjugates are used as NMR contrast agents for biomolecules which are in some cases very specific and whose steady-state concentration in the target tissue is extremely low. In addition, the inventive conjugates show increased relaxivities at high field strengths in the region of 60 MHz, so that they are particularly suitable for use together with instruments used in clinics.

The inventive conjugates additionally show a good solubility in water, and the specificity of the biomolecule is not impaired by the conjugate. The metal complex conjugates are generally just as well tolerated as the unconjugated biomolecule.

The stability of the inventive conjugates is very high so that solutions or freeze-dried products can be stored without loss of activity for a prolonged period. Finally, the complexes display high complex stability, which ensures that no toxic metal is liberated in vivo. This is particularly important because the residence time of such compounds in tissue may be more than 24 hours.

The present invention is explained in detail by the following examples without restricting it thereto.

### EXAMPLES

### Example 1

### a) Benzyl [4-(2-benzyloxycarbonylamino-3-hydroxypropyl)-phenoxy]acetate

60.2 g (0.2 mol) of N-(Z)-tyrosinol (Kashima et al., J. Chem. Soc., Perkin Trans. 1, 1988, 535) and 30.4 g (0.22 mol) of potassium carbonate are dissolved in 500 ml of tetrahydrofuran and 50 ml of water and, at 0° C., a solution of 50.4 g (0.22 mol) of benzyl bromoacetate (Aldrich) in 100 ml of tetrahydrofuran is added dropwise over the course of 30 min, and the mixture is stirred at room temperature for 18 h. The reaction mixture is evaporated to dryness and chromatographed on silica gel (methylene chloride/methanol 10:1). The fractions containing the product are combined and evaporated.

Yield: 63.9 g (71% of theory)

Elemental analysis: C 69.47 (69.64) H 6.05 (6.01) N 3.12 (3.09)

### b) Benzyl (4-{2-benzyloxycarbonylamino-3-[bis(2-benzyloxycarbonylaminoethyl)amino]propyl}phenoxy)acetate

22.48 g (50 mmol) of benzyl [4-(2-benzyloxycarbonylamino-3-hydroxypropyl)phenoxy]acetate are dissolved in 200 ml of methylene chloride and, at −78° C., a solution of 15.5 g (55 mmol) of trifluoromethanesulphonic anhydride (Aldrich) and 6.97 g (65 mmol) of 2,6-dimethylpyridine (Aldrich) in 100 ml of methylene chloride is added dropwise over the course of 30 min, and the mixture is stirred at 0° C. for 3 h. The reaction mixture is extracted twice with 100 ml of ice-water each time, and the organic phase is dried with sodium sulphate. The crude product is then added dropwise at −20° C. to a solution of 18.57 g (50 mmol) of N,N″-di-Z-diethylenetriamine (Fluka) and 12.9 g (100 mmol) of ethyldiisopropylamine in 200 ml of methylene chloride and stirred at −20° C. for 6 h. The mixture is then stirred at room temperature for 24 h. The reaction mixture is extracted twice with 150 ml of water each time, and the organic phase is dried with sodium sulphate, evaporated to dryness and chromatographed on silica gel (hexane/ethyl acetate 5:1). The fractions containing the product are combined and evaporated.

Yield: 16.9 g (42% of theory)

Elemental analysis: C 68.81(69.21) H 6.28 (6.15) N 6.98 (6.90)

### c) (4-{2-Amino-3-[bis(2-aminoethyl)amino]propyl}-phenoxy)acetic acid

16.06 g (200 mmol) of benzyl (4-{2-benzyloxycarbonylamino-3-[bis(2-benzyloxycarbonylaminoethyl)-amino]propyl}phenoxy)acetate are dissolved in 300 ml of isopropanol and mixed with 30 ml of water, and 3 g of palladium catalyst (10% Pd/C) are added. Hydrogenation is carried out at 50° C. for 8 hours. The catalyst is filtered off and the filtrate is evaporated to dryness in vacuo.

Yield: 6.2 g (quantitative) of a colourless powder

Elemental analysis: C 58.04 (58.21) H 8.44 (8.40) N 18.05 (18.01)

### d) {4-(3-[bis(2-{[3-Benzyloxy-1-(2-methoxyethyl)-2-oxo-1,2-dihydropyridine-4-carbonyl]amino}ethyl)amino]-2-{[3-benzyloxy-1-(2-methoxyethyl)-2-oxo-1,2-dihydropyridine-4-carbonyl]amino}propyl)phenoxy)acetic acid

4.65 g (15 mmol) of (4-{2-amino-3-[bis(2-aminoethyl)-amino]propyl}phenoxy)acetic acid and 25.5 g (63 mmol) of 3-benzyloxy-1-(2-methoxyethyl)-4-(2-thioxothiazolidine-3-carbonyl)-1[H]-pyridin-2-one (Raymond et al., Inorg. Chem. (2000), (39), 2652) are dissolved in 200 ml of methylene chloride and stirred at room temperature for three days. The reaction mixture is extracted with 100 ml of 1N sodium hydroxide solution and with 100 ml of saturated sodium chloride solution, and the organic phase is dried with sodium sulphate, evaporated to dryness and chromatographed on silica gel (methylene chloride/methanol 20:1). The fractions containing the product are combined and evaporated.

Yield: 14.5 g (83% of theory)

Elemental analysis: C 64.88 (65.09) H 6.14 (6.08) N 8.41 (8.34)

### e) {4-(3-[bis(2-{[3-Hydroxy-1-(2-methoxyethyl)-2-oxo-1,2-dihydropyridine-4-carbonyl]amino}ethyl)amino]-2-{[3-hydroxy-1-(2-methoxyethyl)-2-oxo-1,2-dihydropyridine-4-carbonyl]amino}propyl)phenoxy)acetic acid

14.0 g (12 mmol) of {4-(3-[bis(2-{[3-benzyloxy-1-(2-methoxyethyl)-2-oxo-1,2-dihydropyridine-4-carbonyl]-amino}ethyl)amino]-2-{[3-benzyloxy-1-(2-methoxyethyl)-2-oxo-1,2-dihydropyridine-4-carbonyl]amino}propyl)phenoxy)-acetic acid are dissolved in 100 ml of acetic acid and 100 ml of concentrated hydrochloric acid and stirred in the dark under nitrogen for three days. This is followed by concentration in vacuo and mixing three times with 200 ml of methanol each time and again concentrating each time. The residue is taken up in 25 ml of methanol and, while stirring, slowly added to 1000 ml of diethyl ether. The precipitated white solid is filtered off with suction, washed with diethyl ether and dried in vacuo.

Yield: 9.9 g (92% of theory)

Elemental analysis: C 56.31 (56.67) H 5.96 (6.01) N 10.94 (10.63)

### f) Gadolinium complex of {4-(3-[bis(2-{[3-hydroxy-1-(2-methoxyethyl)-2-oxo-1,2-dihydropyridine-4-carbonyl]amino}ethyl)amino]-2-{[3-hydroxy-1-(2-methoxyethyl)-2-oxo-1,2-dihydropyridine-4-carbonyl]amino}propyl)phenoxy)acetic acid

8.96 g (10 mmol) of {4-(3-[bis(2-{[3-hydroxyl-1-(2-methoxyethyl)-2-oxo-1,2-dihydropyridine-4-carbonyl]-amino}ethyl)amino]-2-{[3-hydroxy-1-(2-methoxyethyl)-2-oxo-1,2-dihydropyridine-4-carbonyl]amino}propyl)-phenoxy)acetic acid are dissolved in 200 ml of tetrahydrofuran and 40 ml of methanol under reflux, and 2.3 g (10 mmol) of gadolinium trichloride hexahydrate, dissolved in 20 ml of tetrahydrofuran/methanol (5:1), are slowly added while heating, during which a white precipitate separates out. Subsequently, 5 ml of pyridine are added and the mixture is heated under reflux for 18 h. After complexation is complete, the mixture is concentrated in vacuo and chromatographed on silica gel (mobile phase: dichloromethane/methanol/ammonia: 20/20/1). The fractions containing the product are combined and evaporated.

Yield: 7.4 g (67% of theory) of a colourless powder. Water content (Karl-Fischer): 4.1%

Elemental analysis (based on anhydrous substance): C 48.04 (48.11) H 4.80 (4.86) Gd 14.97 (14.66) N 9.34 (9.39)

### Example 2

### a) 1,5-bis[3-Benzyloxy-1-(2-methoxyethyl)-2-oxo-1,2-dihydropyridine-4-carboxamido]-3-azapentane

5.16 g (50 mmol) diethylenetriamine (Fluka) and 40.5 g (100 mmol) of 3-benzyloxy-1-(2-methoxyethyl)-4-(2-thioxothiazolidine-3-carbonyl)-1[H]-pyridin-2-one (Raymond et al., Inorg. Chem. (2000), (39), 2652) are dissolved in 200 ml of methylene chloride and stirred at room temperature for three days. The reaction mixture is extracted with 100 ml of 1N sodium hydroxide solution and with 100 ml of saturated sodium chloride solution, and the organic phase is dried with sodium sulphate, evaporated to dryness and chromatographed on silica gel (methylene chloride/methanol 20:1). The fractions containing the product are combined and evaporated.

Yield: 18.4 g (84% of theory)

Elemental analysis: C 64.18 (64.29) H 6.43 (6.38) N 10.39 (10.35)

### b) Methyl 2-([3-benzyloxy-1-(2-methoxyethyl)-2-oxo-1,2-dihydropyridine-4-carbonyl]amino}-3-(4-tert-butoxycarbonylmethoxyphenoxy)propionate

30.9 g (100 mmol) of methyl 2-amino-3-(4-tert-butoxycarbonylmethoxyphenoxy)propionate (Platzek et al., Schering AG, Germany, Ger. Offen. (1996), 33 pp. CODEN: GWXXBX DE 4425781 A1 19960118) and 42.5 g (105 mmol) of 3-benzyloxy-1-(2-methoxyethyl)-4-(2-thioxothiazolidine-3-carbonyl)-1[H]-pyridin-2-one (Raymond et al., Inorg. Chem. (2000), (39), 2652) are dissolved in 400 ml of methylene chloride and stirred at room temperature for three days. The reaction mixture is extracted twice with 200 ml of saturated sodium bicarbonate solution each time, and the organic phase is dried with sodium sulphate, evaporated to dryness and chromatographed on silica gel (ethyl acetate/hexane 1:5). The fractions containing the product are combined and evaporated.

Yield: 50.1 g (79% of theory)

Elemental analysis: C 64.63 (64.77) H 6.44 (6.38) N 4.71 (4.67)

### c) 2-{[3-Benzyloxy-1-(2-methoxyethyl)-2-oxo-1,2-dihydropyridine-4-carbonyl]-amino)-3-(4-tert-butoxycarbonylmethoxyphenoxy)propionic acid

50.0 g (84.1 mmol) of methyl 2-{[3-benzyloxy-1-(2-methoxyethyl)-2-oxo-1,2-dihydropyridine-4-carbonyl]-amino]-3-(4-tert-butoxycarbonylmethoxyphenoxy)propionate are dissolved in 400 ml of methanol and 100 ml of 2N sodium hydroxide solution and stirred at room temperature for 2 h. The mixture is acidified with 2N HCl solution (pH=3.5) and most of the methanol is distilled off in vacuo. The reaction mixture is mixed with 300 ml of water and extracted twice with 250 ml of ethyl acetate each time, and the organic phase is dried with sodium sulphate, evaporated to dryness and chromatographed on silica gel (ethyl acetate/hexane 1:3). The fractions containing the product are combined and evaporated.

Yield: 43 g (88% of theory)

Elemental analysis: C 64.13 (64.29) H 6.25 (6.19) N 4.82 (4.80)

### d) tert-Butyl {4-(3-[bis(2-{[3-benzyloxy-1-(2-methoxyethyl)-2-oxo-1,2-dihydropyridine-4-carbonyl]amino}-ethyl)carbamoyl]-2-{[3-benzoyloxy-1-(2-methoxyethyl)-2-oxo-1,2-dihydropyridine-4-carbonyl]amino}-ethyl)phenoxy)acetate

17.42 g (30 mmol) of 2-{[3-benzyloxy-1-(2-methoxyethyl)-2-oxo-1,2-dihydropyridine-4-carbonyl)amino-3-(4-tert-butoxycarbonylmethoxyphenoxy)propionic acid and 20.2 g (30 mmol) of 1,5-bis[3-benzyloxy-1-(2-methoxyethyl)-2-oxo-1,2-dihydropyridine-4-carboxamido]-3-aza]pentane are dissolved in 300 ml of tetrahydrofuran and, at 0° C., 18.9 g (36 mmol) of EEDQ [2-ethoxy-1-ethoxycarbonyl-1,2-dihydroquinoline] (Fluka) are added and then stirred at room temperature for 20 h. The reaction mixture is evaporated to dryness and chromatographed on silica gel (methylene chloride/methanol 20:1). The fractions containing the product are combined and evaporated.

Yield: 24.0 g (65% of theory)

Elemental analysis: C 65.09 (65.33) H 6.28 (6.14) N 7.93 (7.88)

### e) {4-(3-[bis(2-{[3-Hydroxy-1-(2-methoxyethyl)-2-oxo-1,2-dihydropyridine-4-carbonyl]amino}ethyl)-carbamoyl]-2-{[3-hydroxy-1-(2-methoxyethyl)-2-oxo-1,2-dihydropyridine-4-carbonyl]amino}ethyl)phenoxy)acetic acid

14.8 g (12 mmol) of tert-butyl {4-(3-[bis(2-{[3-benzyloxy-1-(2-methoxyethyl)-2-oxo-1,2-dihydropyridine-4-carbonyl]amino}ethyl)carbamoyl]-2-{[3-benzyloxy-1-(2-methoxyethyl)-2-oxo-1,2-dihydropyridine-4-carbonyl]-amino}ethyl)phenoxy)acetate are dissolved in 100 ml of acetic acid and 100 ml of concentrated hydrochloric acid and stirred in the dark under nitrogen for three days. This is followed by concentration in vacuo and mixing three times with 200 ml of methanol each time and again concentrating each time. The residue is taken up in 25 ml of methanol and, while stirring, slowly added to 1000 ml of diethyl ether. The precipitated white solid is filtered off with suction, washed with diethyl ether and dried in vacuo.

Yield: 9.8 g (90% of theory)

Elemental analysis: C 55.44 (55.66) H 5.65 (5.60) N 10.78 (10.75)

### f) Gadolinium complex of {4-(3-[bis(2-{[3-hydroxy-1-(2-methoxyethyl)-2-oxo-1,2-dihydropyridine-4-carbonyl]amino}ethyl)carbamoyl]-2-{[3-hydroxy-1-(2-methoxyethyl)-2-oxo-1,2-dihydropyridine-4-carbonyl]amino)ethyl)phenoxy)acetic acid

9.1 g (10 mmol) of {4-(3-[bis(2-{[3-hydroxy-1-(2-methoxyethyl)-2-oxo-1,2-dihydropyridine-4-carbonyl]-amino}ethyl)carbamoyl]-2-{[3-hydroxy-1-(2-methoxyethyl)-2-oxo-1,2-dihydropyridine-4-carbonyl]amino)-ethyl)phenoxy)acetic acid are dissolved in 200 ml of tetrahydrofuran and 40 ml of methanol under reflux, and 2.3 g (10 mmol) of gadolinium trichloride hexahydrate, dissolved in 20 ml of tetrahydrofuran/methanol (5:1), are slowly added while heating, during which a white precipitate separates out. Then 5 ml of pyridine are added and heated under reflux for 18 h. After complexation is complete, the mixture is concentrated in vacuo and chromatographed on silica gel (mobile phase: dichloromethane/methanol/ammonia: 20/20/1). The fractions containing the product are combined and evaporated.

Yield: 6.7 g (61% of theory) of a colourless powder. Water content (Karl-Fischer): 3.8%

Elemental analysis (based on anhydrous substance): C 47.41 (47.56) H 4.55 (4.57) Gd 14.78 (14.69) N 9.21 (9.20)

### Example 3

### a) {4-(3-[bis(2-{[3-Benzyloxy-1-methyl-2-oxo-1,2-dihydropyridine-4-carbonyl]amino}ethyl)amino]-2-{[3-benzyloxy-1-methyl-2-oxo-1,2-dihydropyridine-4-carbonyl]amino}propyl)phenoxy)acetic acid

4.65 g (15 mmol) of (4-{2-amino-3-[bis(2-aminoethyl)-amino]propyl}phenoxy)acetic acid and 22.7 g (63 mmol) of 3-benzyloxy-1-methyl-4-(2-thioxothiazolidine-3-carbonyl)-1[H]-pyridin-2-one (Raymond et al.) are dissolved in 200 ml of methylene chloride and stirred at room temperature for three days. The reaction mixture is extracted with 100 ml of 1N sodium hydroxide solution and with 100 ml of saturated sodium chloride solution, and the organic phase is dried with sodium sulphate, evaporated to dryness and chromatographed on silica gel (methylene chloride/methanol 20:1). The fractions containing the product are combined and evaporated.

Yield: 13.4 g (86% of theory)

Elemental analysis: C 66.20 (66.31) H 5.75 (5.72) N 9.48 (9.44)

### b) {4-(3-[bis(2-{[3-Hydroxy-1-methyl-2-oxo-1,2-dihydropyridine-4-carbonyl]amino}ethyl)amino]-2-{[3-hydroxy-1-methyl-2-oxo-1,2-dihydropyridine-4-carbonyl]amino}propyl)phenoxy)acetic acid

12.4 g (12 mmol) of {4-(3-[bis(2-{[3-benzyloxy-1-methyl-2-oxo-1,2-dihydropyridine-4-carbonyl]amino}ethyl)amino]-2-{[3-benzyloxy-1-methyl-2-oxo-1,2-dihydropyridine-4-carbonyl]amino}propyl)phenoxy)acetic acid are dissolved in 100 ml of acetic acid and 100 ml of concentrated hydrochloric acid and stirred in the dark under nitrogen for three days. This is followed by concentration in vacuo and mixing three times with 200 ml of methanol each time and again concentrating each time. The residue is taken up in 25 ml of methanol and, while stirring, slowly added to 1000 ml of diethyl ether. The precipitated white solid is filtered off with suction, washed with diethyl ether and dried in vacuo.

Yield: 8.2 g (90% of theory)

Elemental analysis: C 56.61 (56.85) H 5.41 (5.33) N 12.84 (12.79)

### c) Gadolinium complex of {4-(3-[bis(2-{[3-hydroxy-1-methyl-2-oxo-1,2-dihydropyridine-4-carbonyl]-amino}ethyl)amino]-2-{[3-hydroxy-1-methyl-2-oxo-1,2-dihydropyridine-4-carbonyl]amino}propyl)phenoxy)-acetic acid

7.64 g (10 mmol) of {4-(3-[bis(2-{[3-hydroxy-1-methyl-2-oxo-1,2-dihydropyridine-4-carbonyl]amino}ethyl)amino]-2-{[3-hydroxy-1-methyl-2-oxo-1,2-dihydropyridine-4-carbonyl]amino}propyl)phenoxy)acetic acid are dissolved in 200 ml of tetrahydrofuran and 40 ml of methanol under reflux, and 2.3 g (10 mmol) of gadolinium trichloride hexahydrate, dissolved in 20 ml of tetrahydrofuran/methanol (5:1), are slowly added while heating, during which a white precipitate separates out. Then 5 ml of pyridine are added and heated under reflux for 18 h. After complexation is complete, the mixture is evaporated in vacuo and chromatographed on silica gel (mobile phase: dichloromethane/methanol/ammonia: 20/20/1). The fractions containing the product are combined and evaporated.

Yield: 5.6 g (59% of theory) of a colorless powder. Water content (Karl-Fischer): 3.7%

Elemental analysis (based on anhydrous substance): C 47.10 (47.43) H 4.17 (4.22) Gd 17.13 (16.88) N 10.68(10.67)

### Example 4

### a) Benzyl 4-benzyloxycarbonylamino-5-[bis(2-benzyloxy-carbonylaminoethyl)amino]pentanecarboxylate

17.87 g (50 mmol) of Z—Glu—(OBn)—OH (Bachem) are dissolved in 200 ml of methylene chloride and, at −78° C., a solution of 15.5 g (55 mmol) of trifluoromethanesulphonic anhydride (Aldrich) and 6.97 g (65 mmol) of 2,6-dimethylpyridine (Aldrich) in 100 ml of methylene chloride is added dropwise over the course of 30 min, and the mixture is stirred at 0° C. for 3 h. The reaction mixture is extracted twice with 100 ml of ice-water each time, and the organic phase is dried with sodium sulphate. The crude product is then added dropwise at −20° C. to a solution of 18.57 g (50 mmol) of N,N″-di-Z-diethylenetriamine (Fluka) and 12.9 g (100 mmol) of ethyldiisopropylamine in 200 ml of methylene chloride and stirred at −20° C. for 6 h. The mixture is then stirred at room temperature for 24 h. The reaction mixture is extracted twice with 150 ml of water each time, and the organic phase is dried with sodium sulphate, evaporated to dryness and chromatographed on silica gel (hexane/ethyl acetate 5:1). The fractions containing the product are combined and evaporated.

Yield: 14.5 g (41% of theory)

Elemental analysis: C 67.21(67.44) H 6.52 (6.49) N 7.88 (7.88)

### b) 4-Amino-5-[bis(2-aminoethyl)amino]pentanecarboxylic acid

14.2 g (20 mmol) of benzyl 4-benzyloxycarbonylamino-5-[bis(2-benzyloxycarbonylaminoethyl)amino]pentanecarboxylate are dissolved in 300 ml of isopropanol and mixed with 30 ml of water, and 3 g of palladium catalyst (10% Pd/C) are added. Hydrogenation is carried out at 50° C. for 8 hours. The catalyst is filtered off, and the filtrate is evaporated to dryness in vacuo.

Yield: 4.35 g (quantitative) of a colourless powder

Elemental analysis: C 49.52 (49.67) H 10.16 (10.18) N 25.67 (25.57)

### c) 5-[bis(2-{[3-Benzyloxy-1-(2-methoxyethyl)-2-oxo-1,2-dihydropyridine-4-carbonyl]amino}ethyl)amino]-4-{[3-benzyloxy-1-(2-methoxyethyl)-2-oxo-1,2-dihydropyridine-4-carbonyl]amino}pentanecarboxylic acid

3.27 g (15 mmol) of 4-amino-5-[bis(2-aminoethyl)amino]-pentanecarboxylic acid and 25.5 g (63 mmol) of 3-benzyloxy-1-(2-methoxyethyl)-4-(2-thioxothiazolidine-3-carbonyl)-1[H]-pyridin-2-one (Raymond et al., Inorg. Chem. (2000), (39), 2652) are dissolved in 200 ml of methylene chloride and stirred at room temperature for three days. The reaction mixture is extracted with 100 ml of 1N sodium hydroxide solution and with 100 ml of saturated sodium chloride solution, and the organic phase is dried with sodium sulphate, evaporated to dryness and chromatographed on silica gel (methylene chloride/methanol 20:1). The fractions containing the product are combined and evaporated.

Yield: 13.7 g (85% of theory)

Elemental analysis: C 63.73 (63.88) H 6.29 (6.30) N 9.13 (9.07)

### d) 5-[bis(2-{[3-Hydroxy-1-(2-methoxyethyl)-2-oxo-1,2-dihydropyridine-4-carbonyl]amino}ethyl)amino]-4-{[3-hydroxy-1-(2-methoxyethyl)-2-oxo-1,2-dihydropyridine-4-carbonyl]amino]pentanecarboxylic acid

12.9 g (12 mmol) of 5-[bis(2-{[3-benzyloxy-1-(2-methoxyethyl)-2-oxo-1,2-dihydropyridine-4-carbonyl]-amino}ethyl)amino]-4-{[3-benzyloxy-1-(2-methoxyethyl)-2-oxo-1,2-dihydropyridine-4-carbonyl]amino]pentanecarboxylic acid are dissolved in 100 ml of acetic acid and 100 ml of concentrated hydrochloric acid and stirred in the dark under nitrogen for three days. This is followed by concentration in vacuo and mixing three times with 200 ml of methanol each time and again concentrating each time. The residue is taken up in 25 ml of methanol and, while stirring, slowly added to 1000 ml of diethyl ether. The precipitated white solid is filtered off with suction, washed with diethyl ether and dried in vacuo.

Yield: 9.1 g (94% of theory)

Elemental analysis: C 53.79 (53.91) H 6.14 (6.10) N 12.20 (12.15)

### e) Gadolinium complex of 5-[bis(2-{[3-hydroxy-1-(2-methoxyethyl)-2-oxo-1,2-dihydropyridine-4-carbonyl]-amino}ethyl)amino]-4-{[3-hydroxy-1-(2-methoxyethyl)-2-oxo-1,2-dihydropyridine-4-carbonyl]-amino}pentanecarboxylic acid

8.04 g (10 mmol) of 5-[bis(2-{[3-hydroxy-1-(2-methoxyethyl)-2-oxo-1,2-dihydropyridine-4-carbonyl]amino}ethyl)amino]-4-{[3-hydroxy-1-(2-methoxyethyl)-2-oxo-1,2-dihydropyridine-4-carbonyl]amino}pentanecarboxylic acid are dissolved in 200 ml of tetrahydrofuran and 40 ml of methanol under reflux, and 2.3 g (10 mmol) of gadolinium trichloride hexahydrate, dissolved in 20 ml of tetrahydrofuran/methanol (5:1), are slowly added while heating, during which a white precipitate separates out. Subsequently, 5 ml of pyridine are added and the mixture is heated under reflux for 18 h. After complexation is complete, the mixture is concentrated in vacuo and chromatographed on silica gel (mobile phase: dichloromethane/methanol/ammonia: 20/20/1). The fractions containing the product are combined and evaporated.

Yield: 6.2 g (61% of theory) of a colourless powder. Water content (Karl-Fischer): 4.8%

Elemental analysis (based on anhydrous substance): C 45.13 (45.25) H 4.84 (4.87) Gd 16.41 (15.21) N 10.23 (10.23)

### Example 5

### a) 5-tert-Butyl 1-methyl 2-{[3-benzyloxy-1-(2-methoxyethyl)-2-oxo-1,2-dihydropyridine-4-carbonyl]amino]-pentanecarboxylate

21.7 g (100 mmol) of 5-tert-butyl 1-methyl 2-amino-pentanedicarboxylate (Bachem) and 42.5 g (105 mmol) of 3-benzyloxy-1-(2-methoxyethyl)-4-(2-thioxothiazolidine-3-carbonyl)-1[H]-pyridin-2-one (Raymond et al., Inorg. Chem. (2000), (39), 2652) are dissolved in 400 ml of methylene chloride and stirred at room temperature for three days. The reaction mixture is extracted twice with 200 ml of saturated sodium bicarbonate solution each time, and the organic phase is dried with sodium sulphate, evaporated to dryness and chromatographed on silica gel (ethyl acetate/hexane 1:5). The fractions containing the product are combined and evaporated.

Yield: 41.8 g (83% of theory)

Elemental analysis: C 62.14 (62.28) H 6.82 (6.77) N 5.57 (5.54)

### b) 5-tert-Butyl 2-{[3-benzyloxy-1-(2-methoxyethyl)-2-oxo-1,2-dihydropyridine-4-carbonyl]amino}pentanedicarboxylate

41.0 g (8.16 mmol) of 5-tert-butyl 1-methyl 2-{[3-benzyloxy-1-(2-methoxyethyl)-2-oxo-1,2-dihydropyridine-4-carbonyl]amino}pentanedicarboxylate are dissolved in 400 ml of methanol and 100 ml of 2N sodium hydroxide solution and stirred at room temperature for 2 h. The mixture is acidified with 2N HCl solution (pH=3.5) and most of the methanol is distilled off in vacuo. The reaction mixture is mixed with 300 ml of water and extracted twice with 250 ml of ethyl acetate each time, and the organic phase is dried with sodium sulphate, evaporated to dryness and chromatographed on silica gel (ethyl acetate/hexane 1:3). The fractions containing the product are combined and evaporated.

Yield: 35.7 g (90% of theory)

Elemental analysis: C 61.46 (61.62) H 6.60 (6.54) N 5.73 (5.70)

### c) tert-Butyl 4-(2-[bis(2-{[3-benzyloxy-1-(2-methoxyethyl)-2-oxo-1,2-dihydropyridine-4-carbonyl]amino}-ethyl)carbamoyl]-2-{[3-benzyloxy-1-(2-methoxyethyl)-2-oxo-1,2-dihydropyridine-4-carbonyl]amino}butyrate

14.66 g (30 mmol) of 5-tert-butyl 2-{[3-benzyloxy-1-(2-methoxyethyl)-2-oxo-1,2-dihydropyridine-4-carbonyl]-amino}pentanedicarboxylate and 20.2 (30 mmol) of 1,5-bis[3-benzyloxy-1-(2-methoxyethyl)-2-oxo-1,2-dihydropyridine-4-carboxamido]-3-azapetane are dissolved in 300 ml of tetrahydrofuran and, at 0° C., 18.9 g (36 mmol) of EEDQ [2-ethoxy-1-ethoxycarbonyl-1,2-dihydroquinoline] (Fluka) are added and then stirred at room temperature for 20 h. The reaction mixture is evaporated to dryness and chromatographed on silica gel (methylene chloride/methanol 20:1). The fractions containing the product are combined and evaporated.

Yield: 20.3 g (59% of theory)

Elemental analysis: C 64.03 (64.21) H 6.43 (6.44) N 8.57 (8.50)

### d) 4-(2-[bis(2-{[3-Hydroxy-1-(2-methoxyethyl)-2-oxo-1,2-dihydropyridine-4-carbonyl]amino}ethyl)carbamoyl]-2-{[3-hydroxy-1-(2-methoxyethyl)-2-oxo-1,2-dihydropyridine-4-carbonyl]amino}butyric acid

13.7 g (12 mmol) of tert-butyl 4-(2-[bis(2-{[3-benzyloxy-1-(2-methoxyethyl)-2-oxo-1,2-dihydropyridine-4-carbonyl]amino}ethyl)carbamoyl]-2-{[3-benzyloxy-1-(2-methoxyethyl)-2-oxo-1,2-dihydropyridine-4-carbonyl]-amino}butyrate are dissolved in 100 ml of acetic acid and 100 ml of concentrated hydrochloric acid and stirred in the dark under nitrogen for three days. This is followed by concentration in vacuo and addition three times of 200 ml of methanol each time and renewed concentration each time. The residue is taken up in 25 ml of methanol and, while stirring, slowly added to 1000 ml of diethyl ether. The precipitated white solid is filtered off with suction, washed with diethyl ether and dried in vacuo.

Yield: 8.9 g (91% of theory)

Elemental analysis: C 52.87 (52.80) H 5.79 (5.77) N 11.99 (12.00)

### e) Gadolinium complex of 4-(2-[bis(2-{[3-hydroxy-1-(2-methoxyethyl)-2-oxo-1,2-diydropyridine-4-carbonyl]amino}ethyl)carbamoyl]-2-[{3-hydroxy-1-(2-methoxyethyl)-2-oxo-1,2-dihydropyridine-4-carbonyl]amino}butyric acid

8.2 g (10 mmol) of 4-(2-[bis(2-{[3-hydroxy-1-(2-methoxyethyl)-2-oxo-1,2-dihydropyridine-4-carbonyl]-amino)ethyl)carbamoyl]-2-{[3-hydroxy-1-(2-methoxyethyl)-2-oxo-1,2-dihydropyridine-4-carbonyl]amino]butyric acid are dissolved in 200 ml of tetrahydrofuran and 40 ml of methanol under reflux, and 2.3 g (10 mmol) of gadolinium trichloride hexahydrate, dissolved in 20 ml of tetrahydrofuran/methanol (5:1), are slowly added while heating, during which a white precipitate separates out. Then 5 ml of pyridine are added and heated under reflux for 18 h. After complexation is complete, the mixture is concentrated in vacuo and chromatographed on silica gel (mobile phase: dichloromethane/methanol/ammonia: 20/20/1). The fractions containing the product are combined and evaporated.

Yield: 5.8 g (57% of theory) of colourless powder. Water content (Karl-Fischer): 4.1%

Elemental analysis (based on anhydrous substance): C 44.48 (44.76) H 4.56 (4.61) Gd 16.18 (15.89) N 10.09 (10.07)

### Example 6

### a) 5-[bis-(2-{[3-Benzyloxy-1-methyl-2-oxo-1,2-dihydropyridine-4-carbonyl]amino}ethyl)amino]-4-{[3-benzyloxy-1-methyl-2-oxo-1,2-dihydropyridine-4-carbonyl]amino}pentanecarboxylic acid

3.27 g (15 mmol) of 4-amino-5-[bis(2-aminoethyl)amino]pentanecarboxylic acid and 22.7 g (63 mmol) of 3-benzyloxy-1-methyl-4-(2-thioxothiazol]idine-3-carbonyl)-1[H]-pyridin-2-one (Raymond et al., Inorg. Chem. (2000), (39), 2652) are dissolved in 200 ml of methylene chloride and stirred at room temperature for three days. The reaction mixture is extracted with 100 ml of 1N sodium hydroxide solution and with 100 ml of saturated sodium chloride solution, and the organic phase is dried over sodium sulphate, evaporated to dryness and chromatographed on silica gel (methylene chloride/methanol 20:1). The fractions containing the product are combined and evaporated.

Yield: 12.3 g (87% of theory)

Elemental analysis: C 65.03 (65.14) H 5.88 (5.85) N 10.41 (10.38)

### b) 5-[bis-(2-{[3-Hydroxy-1-methyl-2-oxo-1,2-dihydropyridine-4-carbonyl]amino}ethyl)amino]-4-{[3-hydroxy-1-methyl-2-oxo-1,2-dihydropyridine-4-carbonyl]amino}pentanecarboxylic acid

11.3 g (12 mmol) of 5-[bis-(2-{[3-benzyloxy-1-methyl-2-oxo-1,2-dihydropyridine-4-carbonyl]amino}ethyl)amino]-4-{[3-benzyloxy-1-methyl-2-oxo-1,2-dihydropyridine-4-carbonyl]amino}pentanecarboxylic acid are dissolved in 100 ml of acetic acid and 100 ml of concentrated hydrochloric acid and stirred in the dark under nitrogen for three days. This is followed by concentration in vacuo and mixing three times with 200 ml of methanol each time and again concentrating each time. The residue is taken up in 25 ml of methanol and, while stirring, slowly added to 1000 ml of diethyl ether. The precipitated white solid is filtered off with suction, washed with diethyl. ether and dried in vacuo.

Yield: 7.4 g (92% of theory)

Elemental analysis: C 53.65 (53.88) H 5.55 (5.52) N 14.60 (14.49)

### c) Gadolinium complex of 5-[bis-(2-{[3-hydroxy-1-methyl-2-oxo-1,2-dihydropyridine-4-carbonyl]amino}ethyl)amino]-4-{[3-hydroxy-1-methyl-2-oxo-1,2-dihydropyridine-4-carbonyl]amino}pentanecarboxylic acid

6.72 g (10 mmol) of 5-[bis-(2-{[3-hydroxy-1-methyl-2-oxo-1,2-dihydropyridine-4-carbonyl]amino}ethyl)amino]-4-{[3-hydroxy-1-methyl-2-oxo-1,2-dihydropyridine-4-carbonyl]amino}pentanecarboxylic acid are dissolved in 200 ml of tetrahydrofuran and 40 ml of methanol under reflux, and 2.3 g (10 mmol) of gadolinium trichloride hexahydrate, dissolved in 20 ml of tetrahydrofuran/methanol (5:1), are slowly added while heating, during which a white precipitate separates out. Subsequently, 5 ml of pyridine are added and the mixture is heated under reflux for 18 h. After complexation is complete, the mixture is concentrated in vacuo and chromatographed on silica gel (mobile phase: dichloromethane/methanol/ammonia: 20/20/1). The fractions containing the product are combined and evaporated.

Yield: 5.2 g (60% of theory) of a colourless powder. Water content (Karl-Fischer): 3.8%

Elemental analysis (based on anhydrous substance): C 43.63 (43.89) H 4.15 (4.17) Gd 19.04 (18.88) N 11.87 (11.89)

### Example 7

### a) tert-Butyl {5-benzyloxycarbonylamino-6-[bis(2-benzyloxycarbonylaminoethyl)amino]hexyl}carbamate

18.32 g (50 mmol) of Z-Lys-(ε-boc)-ol (Ripka et al., Org. Lett. 2001, 2309-2312) are dissolved in 200 ml of methylene chloride and, at −78° C., a solution of 15.5 g (55 mmol) of trifluoromethanesulphonic anhydride (Aldrich) and 6.97 g (65 mmol) of 2,6-dimethylpyridine (Aldrich) in 100 ml of methylene chloride is added dropwise over the course of 30 min, and the mixture is stirred at 0° C. for 3 h. The reaction mixture is extracted twice with 100 ml of ice-water each time, and the organic phase is dried with sodium sulphate. The crude product is then added dropwise at −20° C. to a solution of 18.57 g (50 mmol) of N,N″-di-Z-diethylenetriamine (Fluka) and 12.9 g (100 mmol) of ethyldiisopropylamine in 200 ml of methylene chloride and stirred at −20° C. for 6 h. The mixture is then stirred at room temperature for 24 h. The reaction mixture is extracted twice with 150 ml of water each time, and the organic phase is dried with sodium sulphate, evaporated to dryness and chromatographed on silica gel (hexane/ethyl acetate 5:1). The fractions containing the product are combined and evaporated.

Yield: 15.9 g (44% of theory)

Elemental analysis: C 65.07 (65.23) H 7.42 (7.37) N 9.73 (9.67)

### b) tert-Butyl {5-amino-6-[bis(2-aminoethyl)amino]hexyl}carbamate

14.4 g (20 mmol) of tert-butyl {5-benzyloxycarbonylamino-6-[bis(2-benzyloxycarbonylaminoethyl)amino]hexyl}carbamate are dissolved in 300 ml of isopropanol and mixed with 30 ml of water, and 3 g of palladium catalyst (10% Pd/C) are added. Hydrogenation is carried out at 50° C. for 8 hours. The catalyst is filtered off and the filtrate is evaporated to dryness in vacuo.

Yield: 6.3 g (quantitative) of a colourless powder

Elemental analysis: C 56.75 (56.89) H 11.11 (11.09) N 22.06 (22.01)

### c) tert-Butyl 6-[bis(2-{[3-benzyloxy-1-(2-methoxyethyl)-2-oxo-1,2-dihydropyridine-4-carbonyl]amino}ethyl)amino]-5-{[3-benzyloxy-1-(2-methoxyethyl)-2-oxo-1,2-dihydropyridine-4-carbonyl]amino}hexylcarbamate

4.76 g (15 mmol) of tert-butyl {5-amino-6-[bis(2-aminoethyl)amino]hexyl}carbamate and 25.5 g (63 mmol) of 3-benzyloxy-1-(2-methoxyethyl)-4-(2-thioxothiazolidin-3-carbonyl)-1[H]-pyridin-2-one (Raymond et al., Inorg. Chem. (2000), (39), 2652) are dissolved in 200 ml of methylene chloride and stirred at room temperature for three days. The reaction mixture is extracted with 100 ml of 1N sodium hydroxide solution and with 100 ml of saturated sodium chloride solution, and the organic phase is dried with sodium sulphate, evaporated to dryness and chromatographed on silica gel (methylene chloride/methanol 20:1). The fractions containing the product are combined and evaporated.

Yield: 14.6 g (83% of theory)

Elemental analysis: C 64.49 (64.58) H 6.87 (6.84) N 9.55 (9.49)

### d) 6-[bis(2-{[3-Hydroxy-1-(2-methoxyethyl)-2-oxo-1,2-dihydropyridine-4-carbonyl]amino}ethyl)amino]-5-{[3-hydroxy-1-(2-methoxyethyl)-2-oxo-1,2-dihydropyridine-4-carbonyl]hexylamine

14.1 g (12 mmol) of tert-butyl 6-[bis(2-{[3-benzyloxy-1-(2-methoxyethyl)-2-oxo-1,2-dihydropyridine-4-carbonyl]-amino}ethyl)amino]-5-{[3-benzyloxy-1-(2-methoxyethyl)-2-oxo-1,2-dihydropyridine-4-carbonyl]amino}hexylcarbamate are dissolved in 100 ml of acetic acid and 100 ml of concentrated hydrochloric acid and stirred in the dark under nitrogen for three days. This is followed by concentration in vacuo and mixing three times with 200 ml of methanol each time and again concentrating each time. The residue is taken up in 25 ml of methanol and, while stirring, slowly added to 1000 ml of diethyl ether. The precipitated white solid is filtered off with suction, washed with diethyl ether and dried in vacuo.

Yield: 8.9 g (92% of theory)

Elemental analysis: C 55.35 (55.54) H 6.78 (6.73) N 13.96 (13.88)

### e) Gadolinium complex of 6-[bis(2-{[3-hydroxy-1-(2-methoxyethyl)-2-oxo-1,2-dihydropyridine-4-carbonyl]amino}ethyl)amino]-5-{[3-hydroxy-1-(2-methoxyethyl)-2-oxo-1,2-dihydropyridine-4-carbonyl]hexylamine

8.03 g (10 mmol) of 6-[bis(2-{[3-hydroxy-1-(2-methoxyethyl)-2-oxo-1,2-dihydropyridine-4-carbonyl]amino}ethyl)amino]-5-{[3-hydroxy-1-(2-methoxyethyl)-2-oxo-1,2-dihydropyridine-4-carbonyl]hexylamine are dissolved in 200 ml of tetrahydrofuran and 40 ml of methanol under reflux, and 2.3 g (10 mmol) of gadolinium trichloride hexahydrate, dissolved in 20 ml of tetrahydrofuran/methanol (5:1), are slowly added while heating, during which a white precipitate separates out. Subsequently, 5 ml of pyridine are added and the mixture is heated under reflux for 18 h. After complexation is complete, the mixture is concentrated in vacuo and chromatographed on silica gel (mobile phase: dichloromethane/methanol/ammonia: 20/20/1). The fractions containing the product are combined and evaporated.

Yield: 5.9 g (59% of theory) of a colourless powder. Water content (Karl-Fischer): 4.1%

Elemental analysis (based on anhydrous substance): C 46.43 (46.62) H 5.37 (5.39) Gd 16.43 (16.26) N 11.71 (11.68)

### f) Gadolinium complex of {6-[bis(2-{[3-hydroxy-1-(2-methoxyethyl)-2-oxo-1,2-dihydropyridine-4-carbonyl]-amino}ethyl)amino]-5-{[3-hydroxy-1-(2-methoxyethyl)-2-oxo-1,2-dihydropyridine-4-carbonyl]hexyl}-2-(maleimido)propionamide

2.87 g (3 mmol) of the Gd complex amide described in Example 7e are dissolved in 15 ml of dimethylformamide and, while cooling in ice, 879 mg (3.3 mmol) of N-maleimidopropionic acid N-hydroxysuccinimide ester (Aldrich) and 0.7 ml of N,N-diisopropylethylamine in 10 ml of dimethylformamide are added and stirred at room temperature overnight. The reaction mixture is cooled in an ice bath and filtered, and the filtrate is evaporated to dryness in vacuo. The residue is chromatographed on silica gel (mobile phase: dichloromethane/methanol: 1/1). The fractions containing the product are combined and evaporated.

Yield: 2.0 g (58% of theory) of a colourless powder. Water content (Karl-Fischer): 5.7%

Elemental analysis (based on anhydrous substance): C 47.69 (47.88) H 5.09 (5.11) Gd 14.19 (13.92) N 11.38 (11.33)

### Example 8

### a) Methyl 2-{[3-benzyloxy-1-(2-methoxyethyl)-2-oxo-1,2-dihydropyridine-4-carbonyl]amino}-6-tert-butoxycarbonylaminohexanoate

26 g (100 mmol) of methyl 2-amino-6-tert-butoxycarbonylaminohexanoate (Bachem) and 42.5 g (105 mmol) of 3-benzyloxy-1-(2-methoxyethyl)-4-(2-thioxothiazolidine-3-carbonyl)-1[H]-pyridin-2-one (Raymond et al., Inorg. Chem. (2000), (39), 2652) are dissolved in 400 ml of methylene chloride and stirred at room temperature for three days. The reaction mixture is extracted twice with 200 ml of saturated sodium bicarbonate solution each time, and the organic phase is dried with sodium sulphate, evaporated to dryness and chromatographed on silica gel (ethyl acetate/hexane 1:5). The fractions containing the product are combined and evaporated.

Yield: 44.3 g (81% of theory)

Elemental analysis: C 61.64(61.79) H 7.20 (7.14) N 7.70 (7.72)

### b) 2-{[3-Benzyloxy-1-(2-methoxyethyl)-2-oxo-1,2-dihydropyridine-4-carbonyl]amino}-6-tert-butoxycarbonylaminohexanoic acid

43.0 g (78.8 mmol) of methyl 2-{[3-benzyloxy-1-(2-methoxyethyl)-2-oxo-1,2-dihydropyridine-4-carbonyl]amino}-6-tert-butoxycarbonylaminohexanoate are dissolved in 400 ml of methanol and 100 ml of 2N of sodium hydroxide solution and stirred at room temperature for 2 h. The mixture is acidified with 2N HCl solution (pH=3.5) and most of the methanol is distilled off in vacuo. The reaction mixture is mixed with 300 ml of water and extracted twice with 250 ml of ethyl acetate each time, and the organic phase is dried with sodium sulphate, evaporated to dryness and chromatographed on silica gel (ethyl acetate/hexane 1:3). The fractions containing the product are combined and evaporated.

Yield: 37.3 g (89% of theory)

Elemental analysis: C 61.00 (61.35) H 7.02 (6.89) N 7.90 (7.82)

### c) tert-Butyl {5-[bis(2-{[3-benzyloxy-1-(2-methoxyethyl)-2-oxo-1,2-dihydropyridine-4-carbonyl]-amino}ethyl)carbamoyl]-5-{[3-benzyloxy-1-(2-methoxyethyl)-2-oxo-1,2-dihydropyridine-4-carbonyl]-amino}pentyl}carbamate

15.95 g (30 mmol) of 2-{[3-benzyloxy-1-(2-methoxyethyl)-2-oxo-1,2-dihydropyridine-4-carbonyl]amino}-6-tert-butoxycarbonylaminohexanoic acid and 20.2 g (30 mmol) of 1,5-bis[3-benzyloxy-1-(2-methoxyethyl)-2-oxo-1,2-dihydropyridine-4-carboxamido]-3-azapetane are dissolved in 300 ml of tetrahydrofuran and, at 0° C., 18.9 g (36 mmol) of EEDQ [2-ethoxy-1-ethoxycarbonyl-1,2-dihydroquinoline] (Fluka) are added and then stirred at room temperature for 20 h. The reaction mixture is evaporated to dryness and chromatographed on silica gel (methylene chloride/methanol 20:1). The fractions containing the product are combined and evaporated.

Yield: 22.5 g (63% of theory)

Elemental analysis: C 63.73 (63.89) H 6.62 (6.59) N 9.44 (9.38)

### d) 5-[bis(2-{[3-Hydroxy-1-(2-methoxyethyl)-2-oxo-1,2-dihydropyridine-4-carbonyl]amino}ethyl)carbamoyl]-5-{[3-hydroxy-1-(2-methoxyethyl)-2-oxo-1,2-dihydropyridine-4-carbonyl]amino}pentylamine

14.25 g (12 mmol) of tert-butyl {5-[bis(2-{[3-benzyloxy-1-(2-methoxyethyl)-2-oxo-1,2-dihydropyridine-4-carbonyl]-amino}ethyl)carbamoyl]-5-{[(3-benzyloxy-1-(2-methoxyethyl)-2-oxo-1,2-dihydropyridine-4-carbonyl]amino}pentyl}carbamate are dissolved in 100 ml of acetic acid and 100 ml of concentrated hydrochloric acid and stirred in the dark under nitrogen for three days. This is followed by concentration in vacuo and addition three times of 200 ml of methanol each time and renewed concentration each time. The residue is taken up in 25 ml of methanol and, while stirring, slowly added to 1000 ml of diethyl ether. The precipitated white solid is filtered off with suction, washed with diethyl ether and dried in vacuo.

Yield: 8.8 g (90% of theory)

Elemental analysis: C 54.40 (54.67) H 6.42 (6.39) N 13.72 (13.65)

### e) Gadolinium complex of 5-[bis(2-{[3-hydroxy-1-(2-methoxyethyl)-2-oxo-1,2-dihydropyridine-4-carbonyl]-amino}ethyl)carbamoyl]-5-{[3-hydroxy-1-(2-methoxyethyl)-2-oxo-1,2-dihydropyridine-4-carbonyl]-amino}pentylamine

8.17 g (10 mmol) of 5-[bis(2-{[3-hydroxy-1-(2-methoxyethyl)-2-oxo-1,2-dihydropyridine-4-carbonyl]amino}ethyl)carbamoyl]-5-{[3-hydroxy-1-(2-methoxyethyl)-2-oxo-1,2-dihydropyridine-4-carbonyl]amino}pentylamine are dissolved in 200 ml of tetrahydrofuran and 40 ml of methanol under reflux, and 2.3 g (10 mmol) of gadolinium trichloride hexahydrate, dissolved in 20 ml of tetrahydrofuran/methanol (5:1), are slowly added while heating, during which a white precipitate separates out. Then 5 ml of pyridine are added and heated under reflux for 18 h. After complexation is complete, the mixture is concentrated in vacuo and chromatographed on silica gel (mobile phase: dichloromethane/methanol/ammonia: 20/20/1). The fractions containing the product are combined and evaporated.

Yield: 6.2 g (61% of theory) of a colourless powder. Water content (Karl-Fischer): 3.7%

Elemental analysis (based on anhydrous substance): C 45.76 (45.99) H 5.09 (5.13) Gd 16.19 (15.88) N 11.54 (11.49)

### f) Gadolinium complex of {6-[bis(2-{[3-hydroxy-1-(2-methoxyethyl)-2-oxo-1,2-dihydropyridine-4-carbonyl]amino}ethyl)amino]-5-{[3-hydroxy-1-(2-methoxyethyl)-2-oxo-1,2-dihydropyridine-4-carbonyl]hexylcarbamoyl}methoxyacetic acid

2.91 g (3 mmol) of the Gd complex amide described in Example 8e are dissolved in 15 ml of dimethylformamide and, while cooling in ice, 697 mg (6 mmol) of diglycolic anhydride (Aldrich) and 1.2 ml of N,N-diisopropylethylamine in 10 ml of dimethylformamide are added, and the mixture is stirred at room temperature overnight. The reaction mixture is cooled in an ice bath and filtered, and the filtrate is evaporated to dryness in vacuo. The residue is chromatographed on silica gel (mobile phase: dichloromethane/methanol: 1/1). The fractions containing the product are combined and evaporated.

Yield: 1.45 g (41% of theory) of a colourless powder Water content (Karl-Fischer): 6.4%

Elemental analysis (based on anhydrous substance): C 45.30 (45.52) H 4.91 (4.95) Gd 14.46 (14.22) N 10.31 (10.30)

### Example 9

### a) tert-Butyl 6-[bis(2-{[3-benzyloxy-1-methyl-2-oxo-1,2-dihydropyridine-4-carbonyl]amino}ethyl)amino]-5-{[3-benzyloxy-1-methyl-2-oxo-1,2-dihydropyridine-4-carbonyl]amino}hexylcarbamate

4.76 g (15 mmol) of tert-butyl {5-amino-6-[bis(2-amino-ethyl)amino]hexyl}carbamate and 22.7 g (63 mmol) of 3-benzyloxy-1-methyl-4-(2-thioxothiazolidine-3-carbonyl)-1[H]-pyridin-2-one (Raymond et al., Inorg. Chem. (2000), (39), 2652) are dissolved in 200 ml of methylene chloride and stirred at room temperature for three days. The reaction mixture is extracted with 100 ml of 1N sodium hydroxide solution and with 100 ml of saturated sodium chloride solution, and the organic phase is dried with sodium sulphate, evaporated to dryness and chromatographed on silica gel (methylene chloride/methanol 20:1). The fractions containing the product are combined and evaporated.

Yield: 13.2 g (84% of theory)

Elemental analysis: C 65.75 (65.91) H 6.58 (5.62) N 10.76 (10.68)

### b) 6-[bis(2-{[3-Hydroxy-1-methyl-2-oxo-1,2-dihydropyridine-4-carbonyl]amino}ethyl)amino]-5-{[3-hydroxy-1-methyl-2-oxo-1,2-dihydropyridine-4-carbonyl]amino}hexylamine

12.5 g (12 mmol) of tert-butyl 6-[bis(2-{[3-benzyloxy-1-methyl-2-oxo-1,2-dihydropyridine-4-carbonyl]amino}ethyl)amino]-5-{[3-benzyloxy-1-methyl-2-oxo-1,2-dihydropyridine-4-carbonyl]amino}hexylcarbamate are dissolved in 100 ml of acetic acid and 100 ml of concentrated hydrochloric acid and stirred in the dark under nitrogen for three days. This is followed by concentration in vacuo and addition three times of 200 ml of methanol each time and renewed concentration each time. The residue is taken up in 25 ml of methanol and, while stirring, slowly added to 1000 ml of diethyl ether. The precipitated white solid is filtered off with suction, washed with diethyl ether and dried in vacuo.

Yield: 7.6 g (93% of theory)

Elemental analysis: C 55.51 (55.60) H 6.31 (6.31) N 16.71 (16.64)

### c) Gadolinium complex of 6-[bis(2-{[3-hydroxy-1-methyl-2-oxo-1,2-dihydropyridine-4-carbonyl]-amino}ethyl)amino]-5-{([3-hydroxy-1-methyl-2-oxo-1,2-dihydropyridine-4-carbonyl]amino}hexylamine

6.71 g (10 mmol) of 6-[bis(2-{[3-hydroxy-1-methyl-2-oxo-1,2-dihydropyridine-4-carbonyl]amino}ethyl)amino]-5-{[3-hydroxy-1-methyl-2-oxo-1,2-dihydropyridine-4-carbonyl]amino}hexylamine are dissolved in 200 ml of tetrahydrofuran and 40 ml of methanol under reflux, and 2.3 g (10 mmol) of gadolinium trichloride hexahydrate, dissolved in 20 ml of tetrahydrofuran/methanol (5:1), are slowly added while heating, during which a white precipitate separates out. Then 5 ml of pyridine are added and heated under reflux for 18 h. After complexation is complete, the mixture is concentrated in vacuo and chromatographed on silica gel (mobile phase: dichloromethane/methanol/ammonia: 20/20/1). The fractions containing the product are combined and evaporated.

Yield: 5.1 g (57% of theory) of a colourless powder. Water content (Karl-Fischer): 4.4%

Elemental analysis (based on anhydrous substance): C 45.14 (45.47) H 4.76 (4.80) Gd 19.06 (18.77) N 13.58 (13.45)

### d) Gadolinium complex of 6-[bis(2-{[3-hydroxy-1-methyl-2-oxo-1,2-dihydropyridine-4-carbonyl]amino}ethyl)amino]-5-{[3-hydroxy-1-methyl-2-oxo-1,2-dihydropyridine-4-carbonyl]amino}-6-(isothiocyanato)hexylamine

2.47 g (3 mmol) of the Gd complex amine described in Example 9c are dissolved in a two-phase mixture of 50 ml of water and 50 ml of methylene chloride and, at 0° C., 1.73 g (15 mmol) of thiophosgene are added. The mixture is allowed to warm to room temperature and then stirred at this temperature for two hours. Subsequently the aqueous phase is extracted three times with 50 ml of methylene chloride each time and freeze dried. The residue is chromatographed on silica gel (mobile phase: dichloromethane/methanol: 1/1). The fractions containing the product are combined and concentrated in vacuo.

Yield: 721 mg (27% of theory) of a colourless powder. Water content (Karl-Fischer): 6.1%

Elemental analysis (based on anhydrous substance): C 44.33 (44.71) H 4.30 (4.34) Gd 18.14 (17.88) N 12.92 (12.84)

### Example 10

### Gadolinium complex of {4-(3-[bis(2-{[3-hydroxy-1-(2-methoxyethyl)-2-oxo-1,2-dihydropyridine-4-carbonyl]-amino}ethyl)amino]-2-{[3-hydroxy-1-(2-methoxyethyl)-2-oxo-1,2-dihydropyridine-4-carbonyl]amino}propyl)phenoxy)-N-[2-(aminoethylethyl)maleimido]acetamide

3.15 g (3 mmol) of the Gd complex acid described in Example 1f are dissolved in 15 ml of dimethylformamide and, while cooling in ice, 380 mg (3.3 mmol) of N-hydroxysuccinimide and 681 mg (3.3 mmol) of dicyclohexylcarbodiimide are added, and the mixture is preactivated in ice for 1 hour. Then a mixture of 839 mg (3.3 mmol) of N-(2-aminoethyl)maleimide trifluoroacetate salt (Arano et al., J. Med. Chem., 1996, 39, 3458) and 0.7 ml of N,N-diisopropylethylamine in 10 ml of dimethylformamide is added, and the mixture is stirred at room temperature overnight. The reaction mixture is again cooled in an ice bath and filtered, and the filtrate is evaporated to dryness in vacuo. The residue is chromatographed on silica gel (mobile phase: dichloromethane/methanol: 1/1). The fractions containing the product are combined and evaporated.

Yield: 1.4 g (37% of theory) of a colourless powder. Water content (Karl-Fischer): 6.1%

Elemental analysis (based on anhydrous substance): C 49.18 (49.44) H 4.81 (4.84) Gd 13.41 (13.21) N 10.75 (10.69)

### Example 11

### a) 5-[bis(2-{[3-Benzyloxy-6-methyl-2-oxo-1,2-dihydropyridine-4-carbonyl]amino}ethyl)amino]-4-{[3-benzyloxy-6-methyl-2-oxo-1,2-dihydropyridine-4-carbonyl]amino}pentanecarboxylic acid

3.27 g (15 mmol) of 4-amino-5-[bis(2-aminoethyl)amino]pentanecarboxylic acid and 22.7 g (63 mmol) of 3-benzyloxy-6-methyl-4-(2-thioxothiazolidine-3-carbonyl)-1[H]-pyridin-2-one (Doble et al., Inorg. Chem., 2003, 42, 4935) are dissolved in 200 ml of methylene chloride and stirred at room temperature for three days. The reaction mixture is extracted with 100 ml of 1N sodium hydroxide solution and with 100 ml of saturated sodium chloride solution, and the organic phase is dried with sodium sulphate, evaporated to dryness and chromatographed on silica gel (methylene chloride/methanol 20:1). The fractions containing the product are combined and evaporated.

Yield: 11.6 g (82% of theory)

Elemental analysis: C 65.03 (64.94) H 5.88 (5.77) N 10.41 (10.51)

### b) 5-[bis(2-{[3-Hydroxy-6-methyl-2-oxo-1,2-dihydropyridine-4-carbonyl]amino}ethyl)amino]-4-{[3-hydroxy-6-methyl-2-oxo-1,2-dihydropyridine-4-carbonyl]amino}pentanecarboxylic acid

11.3 g (12 mmol) of 5-[bis(2-{[3-benzyloxy-6-methyl-2-oxo-1,2-dihydropyridine-4-carbonyl]amino}ethyl)amino]-4-{[3-benzyloxy-6-methyl-2-oxo-1,2-dihydropyridine-4-carbonyl]amino}pentanecarboxylic acid are dissolved in 100 ml of acetic acid and 100 ml of concentrated hydrochloric acid and stirred in the dark under nitrogen for 3 days. This is followed by concentration in vacuo and mixing three times with 200 ml of methanol each time and again concentrating each time. The residue is taken up in 25 ml of methanol and, while stirring, slowly added to 1000 ml of diethyl ether. The precipitated white solid is filtered off with suction, washed with diethyl ether and dried in vacuo.

Yield: 6.9 g (85% of theory)

Elemental analysis: C 53.65 (53.52) H 5.55 (5.67) N 14.60 (14.81)

### c) Gadolinium complex of 5-[bis(2-{[3-hydroxy-6-methyl-2-oxo-1,2-dihydropyridine-4-carbonyl]-amino}ethyl)amino]-4-{[3-hydroxy-6-methyl-2-oxo-1,2-dihydropyridine-4-carbonyl]amino}pentanecarboxylic acid

6.72 g (10 mmol) of 5-[bis(2-{[3-hydroxy-6-methyl-2-oxo-1,2-dihydropyridine-4-carbonyl]amino}ethyl)amino]-4-{[3-hydroxy-6-methyl-2-oxo-1,2-dihydropyridine-4-carbonyl]amino}pentanecarboxylic acid are dissolved in 200 ml of tetrahydrofuran and 40 ml of methanol under reflux, and 2.3 g (10 mmol) of gadolinium trichloride hexahydrate, dissolved in 20 ml of tetrahydrofuran/methanol (5:1), are slowly added while heating, during which a white precipitate separates out. Subsequently, 5 ml of pyridine are added and the mixture is heated under reflux for 18 h. After complexation is complete, the mixture is concentrated in vacuo and chromatographed on silica gel (mobile phase: dichloromethane/methanol/ammonia: 20/20/1). The fractions containing the product are combined and evaporated.

Yield: 5.9 g (71% of theory) of powder with a pale greyish yellow colour. Water content (Karl-Fischer): 3.8%

Elemental analysis (based on anhydrous substance):

C 43.63 (43.55) H 4.15 (4.23) Gd 19.04 (18.74) N 11.87 (11.91)

### Example 12

### a) 5-Benzyloxy-2,3-dimethyl-6-(4-nitrophenyloxycarbonyl)-1[H]-pyrimidin-4-one

21.94 g (80 mmol) of 5-benzyloxy-2,3-dimethyl-6-carboxy-1[H]-pyrimidin-4-one (Sunderland et al., Inorg. Chem. 2001, 40, 6746) and 12.24 g (88 mmol) of 4-nitrophenol are dissolved in 600 ml of tetrahydrofuran and, at 0° C., 18.16 g (88 mmol) of dicyclohexylcarbodiimide (Fluka) are added, and the mixture is then stirred at room temperature for 20 h. The reaction mixture is filtered, the filtrate is evaporated to dryness, and the crude product is recrystallized from diisopropyl ether.

Yield: 25.5 g (81% of theory)

Elemental analysis: C 60.76 (60.91) H 4.33 (4.27) N 10.63 (10.42)

### b) 5-[bis(2-{[5-Benzyloxy-2,3-dimethyl-4-oxo-3,4-dihydropyrimidine-6-carbonyl]amino}ethyl)amino]-4-{[5-benzyloxy-2,3-dimethyl-4-oxo-3,4-dihydropyrimidine-6-carbonyl]amino}pentanecarboxylic acid

3.27 g (15 mmol) of 4-amino-5-[bis(2-aminoethyl)amino]pentanecarboxylic acid and 24.91 g (63 mmol) of 5-benzyloxy-2,3-dimethyl-6-(4-nitrophenyloxycarbonyl)-1[H]-pyrimidin-4-one are dissolved in 200 ml of methylene chloride and stirred at room temperature for three days. The reaction mixture is extracted with 100 ml of 1N sodium hydroxide solution and with 100 ml of saturated sodium chloride solution, and the organic phase is dried with sodium sulphate, evaporated to dryness and chromatographed on silica gel (methylene chloride/methanol 20:1). The fractions containing the product are combined and evaporated.

Yield: 9.6 g (65% of theory)

Elemental analysis: C 62.06 (61.93) H 5.92 (5.85) N 14.19 (14.28)

### c) 5-[bis(2-{[5-Hydroxy-2,3-dimethyl-4-oxo-3,4-dihydropyrimidine-6-carbonyl]amino}ethyl)amino]-4-{[5-hydroxy-2,3-dimethyl-4-oxo-3,4-dihydropyrimidine-6-carbonyl]amino}pentanecarboxylic acid

11.8 g (12 mmol) of 5-[bis(2-{[5-benzyloxy-2,3-dimethyl-4-oxo-3,4-dihydropyrimidine-6-carbonyl]amino}ethyl)amino]-4-{[5-benzyloxy-2,3-dimethyl-4-oxo-3,4-dihydropyrimidine-6-carbonyl]amino}pentanecarboxylic acid are dissolved in 100 ml of acetic acid and 100 ml of concentrated hydrochloric acid and stirred in the dark under nitrogen for three days. This is followed by concentration in vacuo and mixing three times with 200 ml of methanol each time and again concentrating each time. The residue is taken up in 25 ml of methanol and, while stirring, slowly added to 1000 ml of diethyl ether. The precipitated white solid is filtered off with suction, washed with diethyl ether and dried in vacuo.

Yield: 7.5 g (87% of theory)

Elemental analysis: C 50.28 (50.02) H 5.63 (5.68) N 19.54 (19.20)

### d) Gadolinium complex of 5-[bis(2-{[5-hydroxy-2,3-dimethyl-4-oxo-3,4-dihydropyrimidine-6-carbonyl]amino}ethyl)amino]-4-{[5-hydroxy-2,3-dimethyl-4-oxo-3,4-dihydropyrimidine-6-carbonyl]amino}pentanecarboxylic acid

7.17 g (10 mmol) of 5-[bis(2-{[5-hydroxy-2,3-dimethyl-4-oxo-3,4-dihydropyrimidine-6-carbonyl]amino}ethyl)amino]-4-{[5-hydroxy-2,3-dimethyl-4-oxo-3,4-dihydropyrimidine-6-carbonyl]amino}pentanecarboxylic acid are dissolved in 200 ml of tetrahydrofuran and 40 ml of methanol under reflux, and 2.3 g (10 mmol) of gadolinium trichloride hexahydrate, dissolved in 20 ml of tetrahydrofuran/methanol (5:1), are slowly added while heating, during which a white precipitate separates out. Subsequently, 5 ml of pyridine are added and the mixture is heated under reflux for 18 h. After complexation is complete, the mixture is concentrated in vacuo and chromatographed on silica gel (mobile phase: dichloromethane/methanol/ammonia: 20/20/1). The fractions containing the product are combined and evaporated.

Yield: 6.9 g (73% of theory) of a greyish yellow powder. Water content (Karl-Fischer): 7.8%

Elemental analysis (based on anhydrous substance): C 41.37 (41.05) H 4.28 (4.22) Gd 18.06 (17.81) N 16.08 (15.84)

### Example 13

### a) {4-(3-[bis(2-{[5-Benzyloxy-2,3-dimethyl-4-oxo-3,4-dihydropyrimidine-6-carbonyl]amino}ethyl)amino]-2-{[5-benzyloxy-2,3-dimethyl-4-oxo-3,4-dihydropyrimidine-6-carbonyl]amino}propyl)phenoxy)acetic acid

4.65 g (15 mmol) of (4-{2-amino-3-[bis(2-aminoethyl)amino]propyl}phenoxy)acetic acid and 23.65 g (63 mmol) of 3-benzyloxy-1,6-dimethyl-4-(2-thioxothiazolidine-3-carbonyl)-1[H]-pyrimidin-2-one (Raymond et al., Inorg. Chem. (2000), (39), 2652) are dissolved in 200 ml of methylene chloride and stirred at room temperature for three days. The reaction mixture is extracted with 100 ml of 1N sodium hydroxide solution and with 100 ml of saturated sodium chloride solution, and the organic phase is dried with sodium sulphate, evaporated to dryness and chromatographed on silica gel (methylene chloride/methanol 20:1). The fractions containing the product are combined and evaporated.

Yield: 12.5 g (77% of theory)

Elemental analysis: C 63.44 (63.31) H 5.79 (5.62) N 12.98 (13.21)

### b) {4-(3-[bis(2-{[5-Hydroxy-2,3-dimethyl-4-oxo-3,4-dihydropyrimidine-6-carbonyl]amino}ethyl)amino]-2-{[5-hydroxy-2,3-dimethyl-4-oxo-3,4-dihydropyrimidine-6-carbonyl]amino}propyl)phenoxy)acetic acid

12.95 g (12 mmol) of {4-(3-[bis(2-{[5-benzyloxy-2,3-dimethyl-4-oxo-3,4-dihydropyrimidine-6-carbonyl]amino}ethyl)amino]-2-{[5-benzyloxy-2,3-dimethyl-4-oxo-3,4-dihydropyrimidine-6-carbonyl]amino}propyl)phenoxy)acetic acid are dissolved in 100 ml of acetic acid and 100 ml of concentrated hydrochloric acid and stirred in the dark under nitrogen for three days. This is followed by concentration in vacuo and mixing three times with 200 ml of methanol each time and again concentrating each time. The residue is taken up in 25 ml of methanol and, while stirring, slowly added to 1000 ml of diethyl ether. The precipitated white solid is filtered off with suction, washed with diethyl ether and dried in vacuo.

Yield: 7.9 g (81% of theory)

Elemental analysis: C 53.46 (53.13) H 5.48 (5.39) N 17.32 (17.27)

### c) Gadolinium complex of {4-(3-[bis(2-{[5-hydroxy-2,3-dimethyl-4-oxo-3,4-dihydropyrimidine-6-carbonyl]amino}ethyl)amino]-2-{[5-hydroxy-2,3-dimethyl-4-oxo-3,4-dihydropyrimidine-6-carbonyl]amino}propyl)phenoxy)acetic acid

7.64 g (10 mmol) of {4-(3-[bis(2-{[5-hydroxy-2,3-dimethyl-4-oxo-3,4-dihydropyrimidine-6-carbonyl]amino}ethyl)amino]-2-{[5-hydroxy-2,3-dimethyl-4-oxo-3,4-dihydropyrimidine-6-carbonyl]amino}propyl)phenoxy)acetic acid are dissolved in 200 ml of tetrahydrofuran and 40 ml of methanol under reflux, and 2.3 g (10 mmol) of gadolinium trichloride hexahydrate, dissolved in 20 ml of tetrahydrofuran/methanol (5:1), are slowly added while heating, during which a white precipitate separates out. Subsequently, 5 ml of pyridine are added and the mixture is heated under reflux for 18 h. After complexation is complete, the mixture is concentrated in vacuo and chromatographed on silica gel (mobile phase: dichloromethane/methanol/ammonia: 20/20/1). The fractions containing the product are combined and evaporated.

Yield: 7.43 g (72% of theory) of a pale yellowish powder. Water content (Karl-Fischer): 6.7%

Elemental analysis (based on anhydrous substance): C 44.90 (45.05) H 4.29 (4.22) Gd 16.33 (15.89) N 14.54 (14.32)

### Example 14

### a) 4-(Amino)-5-(bis{2-[(3-benzyloxy-1-methyl-2-oxo-1,2-dihydropyridine-4-carbonyl) amino]ethyl}amino)pentanecarboxylic acid

3.27 g (15 mmol) of 4-amino-5-[bis(2-aminoethyl)amino]-pentanecarboxylic acid and 10.8 g (30 mmol) of 3-benzyloxy-1-methyl-4-(2-thioxothiazolidine-3-carbonyl)-1[H]-pyridin-2-one (Raymond et al., Inorg. Chem. (2000), (39), 2652) are dissolved in 200 ml of methylene chloride and stirred at room temperature for three days. The reaction mixture is extracted with 100 ml of 1N sodium hydroxide solution and with 100 ml of saturated sodium chloride solution, and the organic phase is dried with sodium sulphate, evaporated to dryness and chromatographed on silica gel (methylene chloride/methanol 10:1). The fractions containing the product are combined and evaporated.

Yield: 4.4 g (42% of theory)

Elemental analysis: C 63.42 (63.52) H 6.33 (6.29) N 11.99 (11.94)

### b) 4-[2,3-Bisbenzyloxy-(4-carboxy)benzoylamino]-5-(bis(2-[(3-benzyloxy-1-methyl-2-oxo-1,2-dihydropyridine-4-carbonyl)amino]ethyl}amino)pentanecarboxylic acid

4.2 g (6 mmol) of 4-(amino)-5-(bis{2-[(3-benzyloxy-1-methyl-2-oxo-1,2-dihydropyridine-4-carbonyl)amino]ethyl}amino)pentanecarboxylic acid and 5.8 g (10 mmol) of (2,3-bisbenzyloxy)-1,4-(bis-2-thioxothiazolidin-3-carbonyl)benzene (Raymond et al., Inorg. Chem. (2003), (42), 4930) are dissolved in 100 ml of methylene chloride and stirred at room temperature for three days. The reaction mixture is extracted with 100 ml of 1N sodium hydroxide solution and with 100 ml of saturated sodium chloride solution, and the organic phase is dried with sodium sulphate, evaporated to dryness and chromatographed on silica gel (methylene chloride/methanol 10:1). The fractions containing the product are combined and evaporated.

Yield: 5.7 g (89% of theory)

Elemental analysis: C 66.78 (66.89) H 5.70 (5.66) N 7.92 (7.95)

### c) 4-[2,3-Dihydroxy-(4-carboxy)benzoylamino]-5-(bis{2-[(3-hydroxy-1-methyl-2-oxo-1,2-dihydropyridine-4-carbonyl)amino]ethyl}amino)pentanecarboxylic acid

5.3 g (5 mmol) of 4-[2,3-bisbenzyloxy-(4-carboxy)benzoylamino]-5-(bis{2-[(3-benzyloxy-1-methyl-2-oxo-1,2-dihydropyridine-4-carbonyl)amino]ethyl}amino)pentanecarboxylic acid are dissolved in 50 ml of acetic acid and 50 ml of concentrated hydrochloric acid and stirred in the dark under nitrogen for three days. This is followed by concentration in vacuo and mixing three times with 100 ml of methanol each time and again concentrating each time. The residue is taken up in 20 ml of methanol and, while stirring, slowly added to 1000 ml of diethyl ether. The precipitated white solid is filtered off with suction, washed with diethyl ether and dried in vacuo.

Yield: 3.15 g (,90% of theory)

Elemental analysis: C 53.14 (53.27) H 5.18 (5.16) N 11.99 (11.95)

### d) Gadolinium complex of 4-[2,3-dihydroxy-(4-carboxy)benzoylamino]-5-(bis{2-[(3-hydroxy-1-methyl-2-oxo-1,2-dihydropyridine-4-carbonyl)amino]ethyl}amino)pentanecarboxylic acid

2.8 g (4 mmol) of 4-[2,3-dihydroxy-(4-carboxy)benzoylamino]-5-(bis{2-[(3-hydroxy-1-methyl-2-oxo-1,2-dihydropyridine-4-carbonyl)amino]ethyl}amino)pentanecarboxylic acid are dissolved in 100 ml of tetrahydrofuran and 20 ml of methanol under reflux, and 0.92 g (4 mmol) of gadolinium trichloride hexahydrate, dissolved in 10 ml of tetrahydrofuran/methanol (5:1), are slowly added while heating, during which a white precipitate separates out. Subsequently, 2 ml of pyridine are added and the mixture is heated under reflux for 18 h. After complexation is complete, the mixture is concentrated in vacuo and chromatographed on silica gel (mobile phase: dichloromethane/methanol/ammonia: 20/20/1). The fractions containing the product are combined and evaporated.

Yield: 2.0 g (65% of theory) of a colourless powder. Water content (Karl-Fischer): 4.1%

Elemental analysis (based on anhydrous substance): C 43.55 (43.69)) H 3.89 (3.92) Gd 18.39 (18.17) N 9.83 (9.81)

### Example 15

### a) 4-(Amino)-5-(bis{2-[(3-benzyloxy-6-methyl-2-oxo-1,2-dihydropyridine-4-carbonyl)amino]ethyl}amino)pentanecarboxylic acid

3.27 g (15 mmol) of 4-amino-5-[bis{2-aminoethyl)amino]pentanecarboxylic acid and 10.8 g (30 mmol) of 3-benzyloxy-6-methyl-4-(2-thioxothiazolidine-3-carbonyl-1[H]-pyridin-2-one (Raymond et al., Inorg. Chem. (2000), (39), 2652) are dissolved in 200 ml of methylene chloride and stirred at room temperature for three days. The reaction mixture is extracted with 100 ml of 1N sodium hydroxide solution and with 100 ml of saturated sodium chloride solution, and the organic phase is dried with sodium sulphate, evaporated to dryness and chromatographed on silica gel (methylene chloride/methanol 10:1). The fractions containing the product are combined and evaporated.

Yield: 4.9 g (47% of theory)

Elemental analysis: C 63.42 (63.49) H 6.33 (6.30) N 11.99 (11.91)

### b) 4-[2,3-Bisbenzyloxy-(4-carboxy)benzoylamino]-5-(bis[2-[(3-benzyloxy-6-methyl-2-oxo-1,2-dihydropyridine-4-carbonyl)amino]ethyl}amino)pentanecarboxylic acid

4.2 g (6 mmol) of 4-(amino)-5-(bis{2-[(3-benzyloxy-6-methyl-2-oxo-1,2-dihydropyridine-4-carbonyl)amino]ethyl}amino)pentanecarboxylic acid and 5.8 g (10 mmol) of (2,3-bisbenzyloxy)-1,4-(bis-2-thioxothiazolidine-3-carbonyl)benzene (Raymond et al., Inorg. Chem. (2003), (42), 4930) are dissolved in 100 ml of methylene chloride and stirred at room temperature for three days. The reaction mixture is extracted with 100 ml of 1N sodium hydroxide solution and with 100 ml of saturated sodium chloride solution, and the organic phase is dried with sodium sulphate, evaporated to dryness and chromatographed on silica gel (methylene chloride/methanol 10:1). The fractions containing the product are combined and evaporated.

Yield: 5.8 g (91% of theory)

Elemental analysis: C 66.78 (66.92) H 5.70 (5.65) N 7.92 (7.88)

### c) 4-[2,3-Dihydroxy-(4-carboxy)benzoylamino]-5-(bis{2-[(3-hydroxy-6-methyl-2-oxo-1,2-dihydropyridine-4-carbonyl)amino]ethyl}amino)pentanecarboxylic acid

5.3 g (5 mmol) of 4-[2,3-bisbenzyloxy-(4-carboxy)benzoylamino]-5-(bis{2-[(3-benzyloxy-6-methyl-2-oxo-1,2-dihydropyridine-4-carbonyl)amino]ethyl}amino)pentanecarboxylic acid are dissolved in 50 ml of acetic acid and 50 ml of concentrated hydrochloric acid and stirred in the dark under nitrogen for three days. This is followed by concentration in vacuo and mixing three times with 100 ml of methanol each time and again concentrating each time. The residue is taken up in 20 ml of methanol and, while stirring, slowly added to 1000 ml of diethyl ether. The precipitated white solid is filtered off with suction, washed with diethyl ether and dried in vacuo.

Yield: 3.0 g (86% of theory)

Elemental analysis: C 53.14 (53.32) H 5.18 (5.24) N 11.99 (11.87)

### d) Gadolinium complex of 4-[2,3-dihydroxy-(4-carboxy)benzoylamino]-5-(bis{2-[(3-hydroxy-6-methyl-2-oxo-1,2-dihydropyridine-4-carbonyl)amino]ethyl}amino)pentanecarboxylic acid

2.8 g (4 mmol) of 4-[2,3-dihydroxy-(4-carboxy)-benzoylamino]-5-(bis{2-[(3-hydroxy-6-methyl-2-oxo-1,2-dihydropyridine-4-carbonyl)amino]ethyl}amino)pentanecarboxylic acid are dissolved in 100 ml of tetrahydrofuran and 20 ml of methanol under reflux, and 0.92 g (4 mmol) of gadolinium trichloride hexahydrate, dissolved in 10 ml of tetrahydrofuran/methanol (5:1), are slowly added while heating, during which a white precipitate separates out. Subsequently, 2 ml of pyridine are added and the mixture is heated under reflux for 18 h. After complexation is complete, the mixture is concentrated in vacuo and chromatographed on silica gel (mobile phase: dichloromethane/methanol/ammonia: 20/20/1). The fractions containing the product are combined and evaporated.

Yield: 2.2 g (61% of theory) of a colourless powder. Water content (Karl-Fischer): 4.3%

Elemental analysis (based on anhydrous substance): C 43.55 (43.59) H 3.89 (3.87) Gd 18.39 (18.22) N 9.83 (9.86)

### Example 16

### a) 1-(Sodiumsulfonatobutyl)-4-carboxy-3-benzyloxy-6-methyl-1[H]-pyridine-2-one

0.41 g (17 mmol) of lithium hydroxide are added to 4.31 g (15 mmol) of 4-ethoxycarbonyl-3-benzyloxy-6-methyl-1[H]-pyridine-2-one (international patent application WO 03/016923, example 2) in 15 ml of DMF and, after addition of 2.04 g (15 mmol) of 1,4-butane sultone, are stirred over night at room temperature. The solvent is then distilled off, 50 ml of 2 N sodium hydroxide solution are added to the residue and stirred at room temperature for six hours. The solution is adjusted to a pH of 3 by adding Amberlite® IR-120 (H+) ion exchanger and freeze-dried. The freeze-dried material is subjected to chromatography on a RP-18-Lichroprep column (eluent: water) . The fractions containing the product are combined and concentrated until dry.

Yield: 2.44 g (39% of the theoretical value)

Elemental analysis: C 51.79 (51.53) H 4.83 (4.97) N 3.36 (3.12) Na 5.51 (5.11) S 7.68 (7.29)

### b) 1-(Sodium sulfonatobutyl)-4-(4-nitrophenyloxycarbonyl)-3-benzyloxy-6-methyl-1[H]-pyridine-2-one

2.09 g (5 mmol) of the title compound from example 16a and 765 mg (5.5 mmol) of nitrophenol are dissolved in 30 ml of DMF, 1 ml of ethyl diisopropyl amine and 1.77 g (5.5 mmol) O-(Benzotriazol-1-yl)-N,N,N′,N′-tetramethyluronium tetrafluoroborate added and stirred over night at room temperature. The reaction mixture is concentrated until dry and subjected to chromatography on silical gel (isopropanol). The fractions containing the product are combined and concentrated.

Yield: 2.02 g (75% of the theoretical value)

Elemental analysis: C 53.53 (53.42) H 4.31 (4.55) N 5.20 (5.03) Na 4.27 (4.02) S 5.95 (6.20)

### c) 5- [bis-(2-{[1-(Sodium sulfonatobutyl)-3-benzyloxy-6-methyl-2-oxo-1,2-dihydropyridine-4-carbonyl]-amino}ethyl)amino]-4-{[1-(sodium sulfonatobutyl)-3-benzyloxy-6-methyl-2-oxo-1,2-dihydropyridine-4-carbonyl]amino}pentane carboxylic acid

2.15 g (4 mmol) of the title compound from example 16b and 262 mg (1.2 mmol) of 4-amino-5-[bis-(2-aminoethyl)amino]pentane carboxylic acid (example 4b) are dissolved in 50 ml of DMF, 870 uL (5 mmol) of ethyl diisopropylamine added and stirred at room temperature for three days. The reaction mixture is concentrated until dry and subjected to chromatography on Lichroprep RP-18 (water/acetonitrile gradient). The fractions containing the product are combined and concentrated.

Yield: 1.51 g (89% of the theoretical value)

Elemental analysis: C 53.42 (53.21) H 5.41 (5.67) N 6.92 (6.77) Na 4.87 (5.01) S 6.79 (6.38)

### d) 5- [bis-(2-{[1-(Sodium sulfonatobutyl)-3-hydroxy-6-methyl-2-oxo-1,2-dihydropyridine-4-carbonyl]amino}ethyl)amino]-4-{[1-(sodium sulfonatobutyl)-3-hydroxy-6-methyl-2-oxo-1,2-dihydropyridine-4-carbonyl]amino}pentane carboxylic acid

1.0 g of palladium catalyst (10% Pd/C) is added to a solution of 1.42 g (1 mmol) of the title compound from example 16c in 100 ml of ethanol and hydrogenated for 48 hrs at room temperature. The catalyst is filtered off and the filtrate concentrated in vacuo until dry. The residue is complexed without further characterisation.

Yield: 1.15 g (quant.)

### e) Gadolinium complex of 5-[bis-(2-{[1-(Sodium sulfonatobutyl)-3-hydroxy-6-methyl-2-oxo-1,2-dihydropyridine-4-carbonyl]-amino}ethyl)amino]-4-{[1-(sodium sulfonatobutyl)-3-hydroxy-6-methyl-2-oxo-1,2-dihydropyridine-4-carbonyl]amino}pentane carboxylic acid

At a pH of 8.5 (pH stat), 371 mg (1 mmol) of gadolinium chloride hexahydrate are added to 1.15 g (1 mmol) of the title compound from example 16d in 50 ml of water and stirred over night at room temperature. The reaction mixture is concentrated until dry and subjected to chromatography on Lichroprep RP-18 (water/acetonitrile gradient). The fractions containing the product are combined and concentrated.

Yield: 1.04 g (72% of the theoretical value) Water content (Karl-Fischer): 8.1%

Elemental analysis (based on the anhydrous substance): C 38.15 (37.88) H 4.12 (4.23) Gd 11.89 (11.62) N 7.41 (7.39) Na 6.95 (7.11) S 7.27 (7.09)

### Example 17

### a) 2,3-Bisbenzyloxyterephthalic acid mono-N-methyl amide

2.73 g (34.9 mmol) of 40% aqueous N-methyl amine solution are added to 20.0 g (34.9 mmol) of disuccinimido-2,3-bis(benzyloxy)terephthalate (J Am Chem Soc 1991, 113, 2965) and stirred over night at room temperature. This is followed by condensation in vacuo and chromatography on silica gel (solvent: dichloromethan/methanol gradient). The fractions containing the product are combined and concentrated.

Yield: 9.7 g (71% of the theoretical value)

Elemental analysis: C 70.58 (70.41) H 5.41 (5.29) N 3.58 (3.62)

### b) 2,3-Bisbenzyloxyterephthalic acid mono-N-methyl amide-mono-(p-nitrophenyl ester)

7.83 g (20 mmol) of the title compound from example 17a and 3.06 g (22 mmol) of nitrophenol are dissolved in 100 ml of DMF, 5 ml of ethyl diisopropyl amine and 7.1 g (22 mmol) of O-(benzotriazol-1-yl)-N,N,N′,N′-tetramethyluronium tetrafluoroborate added and stirred over night at room temperature. The reaction mixture is concentrated until dry and recrystallised from isopropanol.

Yield: 8.40 g (82% of the theoretical value)

Elemental analysis: C 67.96 (67.79) H 4.72 (4.66) N 5.47 (5.53)

### c) 4-Amino-5-[bis-(2-{1-(sodium sulfonatobutyl)-3-benzyloxy-6-methyl-2-oxo-1,2-dihydropyridine-4-carbonyl}-aminoethyl)amino]-pentane carboxylic acid

2.15 g (4 mmol) of the title compound from example 16b and 437 mg (2 mmol) of 4-amino-5-[bis-(2-aminoethyl)amino]pentane carboxylic acid (example 4b) are dissolved in 50 ml of DMF, 870 uL (5 mmol) ethyl diisopropyl amine added and stirred three days at room temperature. The reaction mixture is concentrated until dry and subjected to chromatography on Lichroprep RP-18 (water/acetonitrile gradient). The fractions containing the product are combined and concentrated.

Yield: 1.14 g (56% of the theoretical value)

Elemental analysis: C 53.14 (52.94) H 5.75 (5.67) N 8.26 (8.32) Na 4.52 (4.76) S 6.30 (6.03)

### d) 4-[(2,3-bis(Benzyloxy)-4-methylaminocarbonyl)-benzoylamino]-5-[bis-(2-{[1-(sodium sulfonatobutyl)-3-benzyloxy-6-methyl-2-oxo-1,2-dihydropyridine-4-carbonyl]-amino}ethyl)amino]-pentane carboxylic acid

1.02 g (1 mmol) of the title compound from example 17c and 0.67 g (1.3 mmol) of the activated ester described in example 17b are dissolved in 50 ml of DMF, 1 ml of N-ethyl diisopropyl amine added and stirred for three days at room temperature. The reaction mixture is concentrated until dry and subjected to chromatography on Lichroprep RP-18 (water/acetonitrile gradient). The fractions containing the product are combined and concentrated.

Yield: 1.17 g (84% of the theoretical value)

Elemental analysis: C 58.74 (58.47) H 5.58 (5.67) N 7.05 (7.13) Na 3.31 (3.12) S 4.61 (4.22)

### e) 4-[(2,3-Dihydroxy-4-methylaminocarbonyl)-benzoylamino]-5-[bis-(2-{[1-(sodium sulfonatobutyl)-3-hydroxy-6-methyl-2-oxo-1,2-dihydropyridine-4-carbonyl]-amino}ethyl)amino]-pentane carboxylic acid

0.5 g of palladium catalyst (10% Pd/C) are added to a solution of 973 mg (0.7 mmol) of the title compound from example 17d in 50 ml of ethanol and hydrogenated at room temperature for 48 hrs. The product is filtered off the catalyst and the filtrate concentrated in vacuo until dry. The residue is complexed without further characterisation.

Yield: 720 mg (quant.)

### f) Gadolinium complex of the 4-[(2,3-dihydroxy-4-methylaminocarbonyl)-benzoylamino]-5-[bis-(2-{[1-(sodium sulfonatobutyl)-3-hydroxy-6-methyl-2-oxo-1,2-dihydropyridine-4-carbonyl]-amino}ethyl)amino]-pentane carboxylic acid, sodium salt

At a pH of 8.5 (pH stat), 260 mg (0.7 mmol) of gadolinium chloride hexahydrate are added to 720 mg (0.7 mmol) of the title compound from example 17e in 50 ml of water and stirred over night at room temperature. The reaction mixture is concentrated until dry and subjected to chromatography on Lichroprep RP-18 (water/acetonitrile gradient). The fractions containing the product are combined and concentrated.

Yield: 755 mg (79% of the theoretical value) Water content (Karl-Fischer): 10.0%

Elemental analysis (based on the anhydrous substance): C 39.12 (39.34) H 3.94 (4.07) Gd 12.80 (12.82) N 7.98 (8.21) Na 7.49 (7.86) S 5.22 (5.03)

### Example 18

### a) 1-(Sodium sulfonatopropyl)-4-carboxy-3-benzyloxy-6-methyl-1[H]-pyridine-2-one

0.41 g (17 mmol) of lithium hydroxide are added to 4.31 g (15 mmol) of 4-ethoxycarbonyl-3-benzyloxy-6-methyl-1[H]-pyridine-2-one (international patent application WO 03/016923, example 2) in 15 ml of DMF and, after addition of 1.83 g (15 mmol) of 1,3-propane sultone, stirred over night at room temperature. The solvent is then distilled off, 50 ml of 2 N sodium hydroxide solution are added to the residue and stirred at room temperature for six hours. By adding Amberlite® IR-120 (H⁺) ion exchanger, the solution is adjusted to pH 3 and freeze-dried. The freeze-dried material is subjected to chromatography on a RP-18-Lichroprep column (eluent: water). The fractions containing the product are combined and concentrated until dry.

Yield: 5.49 g (37% of the theoretical value)

Elemental analysis: C 50.62 (50.41) H 4.50 (4.77) N 3.47 (3.30) Na 5.70 (5.92) S 7.95 (7.51)

### b) 1-(Sodium sulfonatopropyl)-4-(4-nitrophenyloxycarbonyl)-3-benzyloxy-6-methyl-1[H]-pyridine-2-one

2.02 g (5 mmol) of the title compound from example 18a and 765 mg (5.5 mmol) of riitrophenol are dissolved in 30 ml of DMF, 1 ml of ethyl diisopropylamine and 1.77 g (5.5 mmol) of O-(benzotriazol-1-yl)-N,N,N′,N′-tetramethyluronium tetrafluoroborate added and stirred over night at room temperature. The reaction mixture is concentrated until dry and subjected to chromatography (isopropanol) on silica gel. The fractions containing the product are combined and concentrated.

Yield: 2.02 g (77% of the theoretical value)

Elemental analysis: C 52.67 (52.55) H 4.04 (3.89) N 5.34 (5.67) Na 4.38 (4.05) S 6.11 (6.49)

### c) 5- [bis-(2-{[1-(Sodium sulfonatopropyl)-3-benzyloxy-6-methyl-2-oxo-1,2-dihydropyridine-4-carbonyl]amino}ethyl)amino]-4-{[1-(sodium sulfonatopropyl)-3-benzyloxy-6-methyl-2-oxo-1,2-dihydropyridine-4-carbonyl]amino}pentane carboxylic acid

2.10 g (4 mmol) of the title compound from example 18b and 262 mg (1.2 mmol) of 4-amino-5-[bis-(2-aminoethyl)amino]pentane carboxylic acid (example 4b) are dissolved in 50 ml of DMF, 870 uL (5 mmol) ethyldiisopropyl amine added and stirred for three days at room temperature. The reaction mixture is concentrated until dry and subjected to chromatography on Lichroprep RP-18 (water/acetonitrile gradient). The fractions containing the product are combined and concentrated.

Yield: 1.35 g (82% of the theoretical value)

Elemental analysis: C 52.43 (52.21) H 5.13 (5.43) N 7.13 (6.96) Na 5.02 (5.00) S 7.00 (6.69)

### d) 5-[bis-(2-{[1-(Sodium sulfonatopropyl)-3-hydroxy-6-methyl-2-oxo-1,2-dihydropyridine-4-carbonyl]amino}ethyl)amino]-4-{[1-(sodium sulfonatopropyl)-3-hydroxy-6-methyl-2-oxo-1,2-dihydropyridine-4-carbonyl]amino}pentane carboxylic acid

1.0 g of palladium catalyst (10% Pd/C) is added to a solution of 1.37 g (1 mmol) of the title compound from example 18c in 100 ml of ethanol and hydrogenated at room temperature for 48 hrs. The material is filtered off the catalyst and the filtrate concentrated in vacuo until dry. The residue is complexed without further characterisation.

Yield: 1.10 g (quant.)

### e) Gadolinium complex of the 5-[bis-(2-{[1-(Sodium sulfonatopropyl)-3-hydroxy-6-methyl-2-oxo-1,2-dihydropyridine-4-carbonyl]-amino}ethyl)amino]-4-{[1-(sodium sulfonatopropyl)-3-hydroxy-6-methyl-2-oxo-1,2-dihydropyridine-4-carbonyl]amino}pentane carboxylic acid

At a pH of 8.5 (pH-stat), 371 mg (1 mmol) of gadoliniumchloride hexahydrate are added to 1.10 g (1 mmol) of the title compound from example 18d in 50 ml of water and stirred over night at room temperature. The reaction mixture is concentrated until dry and subjected to chromatography on Lichroprep RP-18 (water/acetonitrile gradient). The fractions containing the product are combined and concentrated.

Yield: 1.03 g (75% of the theoretical value) Water content (Karl-Fischer): 7.0%

Elemental analysis (based on the anhydrous substance): C 36.59 (36.33) H 3.78 (3.87) Gd 12.28 (12.04) N 7.66 (7.50) Na 7.18 (6.82) S 7.51 (7.62)

### Example 19

### a) 4-Amino-5-[bis-(2-{1-(sodium sulfonatopropyl)-3-benzyloxy-6-methyl-2-oxo-1,2-dihydropyridine-4-carbonyl}-aminoethyl)amino]-pentane carboxylic acid

2.10 g (4 mmol) of the title compound from example 18b and 437 mg (2 mmol) of 4-amino-5-[bis-(2-aminoethyl)amino]pentane carboxylic acid (example 4b) are dissolved in 50 ml of DMF, 870 uL (5 mmol) of ethyl diisopropyl amine added and stirred for three days at room temperature. The reaction mixture is concentrated until dry and subjected to chromatography on Lichroprep RP-18 (water/acetonitrile gradient). The fractions containing the product are combined and concentrated.

Yield: 1.03 g (52% of the theoretical value)

Elemental analysis: C 52.22 (52.13) H 5.50 (5.67) N 8.50 (8.38) Na 4.65 (4.86) S 6.48 (6.17)

### b) 4-[(2,3-bis(benzyloxy)-4-Methylaminocarbonyl)-benzoylamino]-5-[bis-(2-{[1-(sodium sulfonatopropyl)-3-benzyloxy-6-methyl-2-oxo-1,2-dihydropyridine-4-carbonyl]-amino}ethyl)amino]-pentane carboxylic acid

989 mg (1 mmol) of the title compound from example 19a and 0.67 g (1.3 mmol) of the activated ester described in example 17b are dissolved in 50 ml of DMF, 1 ml of N-ethyl diisopropyl amine added and stirred for three days at room temperature. The reaction mixture is concentrated until dry and subjected to chromatography on Lichroprep RP-18 (water/acetonitrile gradient). The fractions containing the product are combined and concentrated.

Yield: 1.08 g (79% of the theoretical value)

Elemental analysis: C 58.18 (58.35) H 5.40 (5.61) N 7.20 (7.06) Na 3.37 (3.20) S 4.71 (4.44)

### c) 4-[(2,3-Dihydroxy-4-methylaminocarbonyl)-benzoylamino]-5- [bis-(2-{[1-(sodium sulfonatopropyl)-3-hydroxy-6-methyl-2-oxo-1,2-dihydropyridine-4-carbonyl]-amino}ethyl)amino]-pentane carboxylic acid

0.5 g of palladium catalyst (10% Pd/C) are added to a solution of 954 mg (0.7 mmol) of the title compound from example 19b in 50 ml of ethanol and hydrogenated for 48 hrs at room temperature. The material is filtered off the catalyst and the filtrate concentrated in vacuo until dry. The residue is complexed without further characterisation.

Yield: 701 mg (quant.)

Elemental analysis: C 45.55 (45.32) H 4.93 (5.12) N 9.79 (9.60) Na 4.59 (4.76) S 6.40 (6.17)

### d) Gadolinium complex of the 4-[(2,3-Dihydroxy-4-methylaminocarbonyl)-benzoylamino]-5-[bis-(2-{[1-(sodium sulfonatopropyl)-3-hydroxy-6-methyl-2-oxo-1,2-dihydropyridine-4-carbonyl]-amino}ethyl)amino]-pentane carboxylic acid

At a pH of 8.5 (pH stat), 260 mg (0.7 mmol) of gadolinium chloride hexahydrate are added to 701 mg (0.7 mmol) of the title compound from example 19c in 50 ml of water and stirred over night at room temperature. The reaction mixture is concentrated until dry and subjected to chromatography on Lichroprep RP-18 (water/acetonitrile gradient). The fractions containing the product are combined and concentrated.

Yield: 700 mg (75% of the theoretical value) Water content (Karl-Fischer): 10%

Elemental analysis (based on the anhydrous substance): C 38.03 (37.88) H 3.70 (3.67) Gd 13.10 (12.96) N 8.17 (8.31) Na 7.66 (7.39) S 5.34 (5.61)

### Example 20

### a) Trifluormethane sulfonic acid-[1,3-bis-(2-benzyloxy-1-benzyloxymethyl-ethoxy)-prop-2-yl]-ester

30.02 g (50 mmol) of 1,3-bis-(2-benzyloxy-1-benzyloxymethyl-ethoxy)-propan-2-ol (Cassel et al., Eur. J. Org. Chem., 2001, 5, 875-896) and 6.43 g (60 mmol) of 2,6-dimethylpyridine are dissolved in 300 ml of methylene chloride, 15.52 g (55 mmol) of trifluormethane sulfonic acid anhydride slowly added at −20° C. and stirred for 2 hrs at this temperature. The reaction mixture is heated to 0° C., extracted twice with 100 ml of ice water each, the organic phase dried with sodium sulfate, concentrated in vacuo until dry and subjected to chromatography on silica gel (hexane/ethyl acetate 10:1). The fractions containing the product are combined and concentrated in vacuo.

Yield: 30.2 g (82% of the theoretical value) as a colourless oil

Elemental analysis: C 62.28 (62.66) H 5.91 (6.12) F 7.78 (7.44)

### b) 3-Benzyloxy-1-[1,3-bis-(2-benzyloxy-1-benzyloxymethyl-ethoxy)-prop-2-yl]-6-methyl-2-oxo-1,2-dihydropyridine-4-carboxylic acid ethyl ester

11.5 g (40 mmol) of 3-benzyloxy-6-methyl-2-oxo-1,2-dihydropyridine-4-carboxylic acid ethyl ester (Doble et al., Inorg. Chem. 2003, 42, 4935) are dissolved at 0° C. in 150 ml of THF and 41.5 ml (44 mmol) of LiHMDS (1.06 M in THF) added slowly. 29.31 g (40 mmol) of the title compound from example 20a dissolved in 100 ml of THF are then dropped in at −20° C. and stirred at this temperature for 2 hrs and at 0° C. for 4 hrs. The reaction mixture is concentrated in vacuo until dry and subjected to chromatography on silica gel (methylene chloride/methanol 20:1). The fractions containing the product are combined and concentrated in vacuo.

Yield: 23.4 g (67% of the theoretical value) as a colourless oil

Elemental analysis: C 73.17 (73.44) H 6.84 (6.87) N 1.61 (1.59)

### c) 3-Benzyloxy-1-[1,3-bis-(2-benzyloxy-1-benzyloxymethyl-ethoxy)-prop-2-yl]-6-methyl-2-oxo-1,2-dihydropyridine-4-carboxylic acid

22.5 g (25.86 mmol) of the title compound from example 20b are dissolved in 200 ml of methanol and 50 ml of 2 N potassium hydroxide solution and heated at reflux for 6 hrs. The mixture is concentrated until dry, the residue taken up in 250 ml of water and acidified with 6 N hydrochloric acid (pH=1). The precipitate is removed by suction, washed several times with water and dried in vacuo.

Yield: 21.4 g (98% of the theoretical value) as a colourless solid

Elemental analysis: C 72.75 (72.88) H 6.58 (6.62) N 1.66 (1.58)

### d) 3-Benzyloxy-1-(1,3-bis-(2-benzyloxy-1-benzyloxymethyl-ethoxy)-prop-2-yl]-6-methyl-4-nitrophenyloxycarbonyl-2-oxo-1,2-dihydropyridine

20.8 g (24.7 mmol) of the title compound from example 20c and 3.78 g (27.17 mmol) of 4-nitrophenol are dissolved in 200 ml of tetrahydrofuran, 5.61 g (27.17 mmol) of dicyclohexylcarbodiimide (Fluka) added at 0° C. and then stirred at room temperature for 20 hrs. The reaction mixture is filtered off, the filtrate concentrated until dry and the crude product recrystallised from diisopropyl ether.

Yield: 20.5 g (87% of the theoretical value) as a colourless solid

Elemental analysis: C 71.09 (71.25) H 6.07 (6.11) N 2.91 (2.87)

### e) [1,3-bis-(2-Benzyloxy-1-benzyloxymethyl-ethoxy)-prop-2-yl]-amine

14.66 g (20 mmol) of the title compound from example 20a are dissolved in 200 ml of dimethyl acetamide, 1.44 g (22 mmol) of sodium azide in 20 ml water added and then stirred for 12 hrs at 60° C. After cooling, the reaction mixture is diluted with 800 ml of water and extracted twice with 300 ml of diethyl ether each. The combined organic phases are dried with sodium sulfate and concentrated until dry. The crude product is dissolved in 500 ml of diethyl ether, 1.9 g (50 mmol) of lithium aluminium hydride are added carefully and stirred for 4 hrs at room temperature. Excess hydride is destroyed by the careful addition of 30 ml of ethanol and 500 ml of water and the mixture then extracted twice with 300 ml of diethyl ether each. The combined organic phases are dried with sodium sulfate, concentrated until dry and the residue subjected to chromatography on silica gel (methylene chloride/methanol 20:1). The fractions containing the product are combined and concentrated in vacuo.

Yield: 8.6 g (71% of the theoretical value) as a colourless oil

Elemental analysis: C 74.10 (74.35) H 7.56 (7.59) N 2.34 (2.27)

### f) 2,3-Bisbenzyloxy-N-[1,3-bis-(2-benzyloxy-1-benzyloxymethyl-ethoxy)-prop-2-yl]-terephthalic acid monoamide methyl ester

2.2 ml (25 mmol) of oxalyl chloride and a drop of DMF are added to a solution of 5.89 g (15 mmol) of 2,3-bisbenzyloxy terephthalic acid monomethyl ester (Doble et al., Inorg. Chem. 2003, 42, 4935) in 50 ml of toluene and the mixture is heated to 60° C. for four hrs. After concentration in vacuo, the residue is dissolved in 30 ml of THF, dropped into a solution of 9.6 g (16 mmol) of the title compound from example 20e cooled to 0° C. and 2.05 g (20 mmol) of triethyl amine and stirred for 16 hrs at room temperature. The mixture is then concentrated in vacuo until dry and the residue subjected to chromatography on silica gel (methylene chloride/methanol 20:1). The fractions containing the product are combined and concentrated in vacuo.

Yield: 12.9 g (89% of the theoretical value) as a colourless oil

Elemental analysis: C 73.98 (74.21) H 6.52 (6.48) N 1.44 (1.37)

### g) 2,3-Bisbenzyloxy-N-[1,3-bis-(2-benzyloxy-1-benzyloxymethyl-ethoxy)-prop-2-yl]-terephthalic acid monoamide

12.5 g (12.83 mmol) of the title compound from example 20f are dissolved in 100 ml of methanol and 25 ml 2 N of potassium hydroxide solution and heated at reflux for 6 hrs. The mixture is concentrated until dry, the residue taken up in 250 ml of water and acidified with 6 N hydrochloric acid (pH=1). The precipitate is removed by suction, washed several times with water and dried in vacuo.

Yield: 11.8 g (96% of the theoretical value) as a colourless solid

Elemental analysis: C 73.81 (73.96) H 6.40 (6.42) N 1.46 (1.44)

### h) 2,3-Bisbenzyloxy-N-[1,3-bis-(2-benzyloxy-1-benzyloxymethyl-ethoxy)-prop-2-yl]-terephthalic acid monoamide-(4-nitrophenyl) ester

11.5 g (11.98 mmol) of the title compound from example 20 g and 1.83 g (13.18 mmol) of 4-nitrophenol are dissolved in 200 ml of tetrahydrofuran, 2.72 g (27.17 mmol) of dicyclohexyl carbodiimide (Fluka) added at 0° C. and then stirred at room temperature for 20 hrs. The reaction mixture is filtered off, the filtrate concentrated until dry and the crude product recrystallised from diisopropyl ether.

Yield: 10.7 g (82% of the theoretical value) as a colourless solid

Elemental analysis: C 72.21 (72.39) H 5.97 (6.00) N 2.59 (2.56)

### i) 4-(Amino)-5-[bis-(2-{3-benzyloxy-1-[1,3-bis-(2-benzyloxy-1-benzyloxymethyl-ethoxy)-prop-2-yl]-6-methyl-2-oxo-1,2-dihydropyridine-4-carbonyl}-aminoethyl)amino]-pentane carboxylic acid

1.09 g (5 mmol) of 4-amino-5-[bis-(2-aminoethyl)-amino]-pentane carboxylic acid and 9.63 g (10 mmol) of the title compound from example 20d are dissolved in 100 ml of methylene chloride and stirred for three days at room temperature. The reaction mixture is extracted with 50 ml of 1 N sodium hydroxide solution and with 50 ml of saturated sodium chloride solution, the organic phase dried with sodium sulfate, concentrated until dry and subjected to chromatography on silica gel (methylene chloride/methanol 10:1). The fractions containing the product are combined and concentrated.

Yield: 4.37 g (47% of the theoretical value)

Elemental analysis: C 71.44 (71.62) H 6.91 (6.97) N 4.50 (4.41)

### j) 4-({2,3-Bisbenzyloxy-N-[1,3-bis-(2-benzyloxy-1-benzyloxymethyl-ethoxy)-prop-2-yl]-ethylcarbamoyl}-benzoylamino)-5-[bis-(2-{3-benzyloxy-1-[1,3-bis-(2-benzyloxy-1-benzyloxymethyl-ethoxy)-prop-2-yl]-6-methyl-2-oxo-1,2-dihydropyridine-4-carbonyl}-aminoethyl]-amino)-pentane carboxylic acid

4.0 g (2.14 mmol) of the title compound from example 20i and 2.89 g (2.67 mmol) of the title compound from example 20h are dissolved in 500 ml of methylene chloride and stirred for three days at room temperature. The reaction mixture is extracted with 50 ml 1 N of sodium hydroxide solution and with 50 ml of saturated sodium chloride solution, the organic phase dried with sodium sulfate, concentrated until dry and subjected to chromatography on silica gel (methylene chloride/methanol 10:1). The fractions containing the product are combined and concentrated.

Yield: 5.66 g (94% of the theoretical value)

Elemental analysis: C 72.71 (72.93) H 6.71 (6.77) N 3.49 (3.44)

### k) 4-({2,3-Dihydroxy-N-[1,3-bis-(2-hydroxy-1-hydroxymethyl-ethoxy)-prop-2-yl]-ethylcarbamoyl}-benzoylamino)-5-[bis-(2-{3-hydroxy-1-[1,3-bis-(2-hydroxy-1-hydroxymethyl-ethoxy)-prop-2-yl]-6-methyl-2-oxo-1,2-dihydropyridine-4-carbonyl}-aminoethyl]-amino)-pentane carboxylic acid

1.0 g of palladium catalyst (10% Pd/C) is added to a solution of 5.0 g (1.78 mmol) of the title compound from example 20j in 100 ml of ethanol and hydrogenated for 48 hrs at room temperature. The mixture is filtered off the catalyst and the filtrate concentrated in vacuo until dry.

Yield: 2.45 g (quantitative) eines farblosen Feststoffs

Elemental analysis: C 50.98 (51.26) H 6.71 (6.78) N 7.18 (7.04)

### l) Gadolinium complex of the 4-({2,3-dihydroxy-N-[1,3-bis-(2-hydroxy-1-hydroxymethyl-ethoxy)-prop-2-yl]-ethylcarbamoyl}-benzoylamino)-5-[bis-(2-{3-hydroxy-1-[1,3-bis-(2-hydroxy-1-hydroxymethyl-ethoxy)-prop-2-yl]-6-methyl-2-oxo-1,2-dihydropyridine-4-carbonyl}-aminoethyl]-amino)-pentane carboxylic acid

2.1 g (1.54 mmol) of the title compound from example 20k are dissolved at reflux in 40 ml of tetrahydrofuran and 10 ml of methanol and 0.36 g (1.55 mmol) of gadolinium trichloride hexahydrate dissolved in 50 ml of tetrahydrofuran/methanol (5:1) slowly added with heat, resulting in precipitation of a white precipitate. Then 5 ml of pyridine are added and heated at reflux for 18 hrs. After complexing has been completed, the mixture is concentrated in vacuo and subjected to chromatography on silica gel (solvent: dichloromethane/methanol/ammonia: 20/20/1). The fractions containing the product are combined and concentrated.

Yield: 1.67 g (67% of the theoretical value) of a powder with a greyish-yellow tinge Water content (Karl-Fischer): 4.7%

Elemental analysis (based on the anhydrous substance): C 45.16 (45.47) H 5.68 (5.81) Gd 10.19 (10.00) N 6.36 (6.22)

### Example 21

### a) 3-Benzyloxy-6-formyl-2-oxo-1,2-dihydropyridine-4-carboxylic acid ethyl ester

5.75 g (20 mmol) of 3-benzyloxy-6-methyl-2-oxo-1,2-dihydropyridine-4-carboxylic acid ethyl ester (Doble et al., Inorg. Chem. 2003, 42, 4935) are dissolved in 50 ml of dioxan and 8.88 g (80 mmol) of selenium oxide added, followed by heating to 100° C. for 60 mins. The reaction mixture is filtered off, the filtrate concentrated in vacuo until dry and subjected to chromatography on silica gel (methylene chloride/methanol 20:1). The fractions containing the product are combined and concentrated in vacuo.

Yield: 5.05 g (84% of the theoretical value) as a yellow solid

Elemental analysis: C 63.78 (64.02) H 5.02 (5.17) N 4.65 (4.33)

### b) 3-Benzyloxy-6-carboxylato-2-oxo-1,2-dihydropyridine-4-carboxylic acid ethyl ester

4.5 g (14.94 mmol) of the title compound from example 21a are dissolved in 50 ml of DMF and 9.22 g (15 mmol) of oxone added, followed by 24 hours of stirring at room temperature. 200 ml of 0.5 M hydrochloric acid are added to the reaction mixture, followed by extraction three times with 100 ml of dichloromethane each. The combined organic phases are dried with magnesium sulfate, concentrated in vacuo until dry and the residue subjected to chromatography on silica gel (methylene chloride/methanol 10:1). The fractions containing the product are combined and concentrated in vacuo.

Yield: 3.04 g (64% of the theoretical value) as a colourless solid

Elemental analysis: C 60.57 (60.72) H 4.77 (4.81) N 4.41 (4.27)

### c) 3-Benzyloxy-6-{[1,3-bis-(2-benzyloxy-1-benzyloxymethyl-ethoxy)-prop-2-yl]-carbamoyl}-2-oxo-1,2-dihydropyridine-4-carboxylic acid ethyl ester

2.2 ml (25 mmol) of oxalyl chloride and a drop of DMF are added to a solution of 4.76 g (15 mmol) of the title compound from example 21b in 50 ml of toluene and heated for 4 hrs to 60° C. After concentration in vacuo, the residue is dissolved in 30 ml of THF and dropped into a solution of 9.6 g (16 mmol) of the title compound from example 20e cooled to 0° C. and 2.05 g (20 mmol) of triethyl amine and stirred for 16 hrs at room temperature. The mixture is then concentrated in vacuo until dry and the residue subjected to chromatography on silica gel (methylene chloride/methanol 20:1). The fractions containing the product are combined and concentrated in vacuo.

Yield: 13.5 g (75% of the theoretical value) as a colourless oil

Elemental analysis: C 70.81 (70.99) H 6.50 (6.53) N 3.12 (3.17)

### d) 3-Benzyloxy-6-{[1,3-bis-(2-benzyloxy-1-benzyloxymethyl-ethoxy)-prop-2-yl]-carbamoyl}-2-oxo-1,2-dihydropyridine-4-carboxylic acid

13.0 g (14.46 mmol) of the title compound from example 21c are dissolved in 100 ml of methanol and 30 ml 2 N potassium hydroxide solution and heated at reflux for 6 hrs. The mixture is concentrated until dry, the residue taken up in 250 ml of water and acidified with 6 N of hydrochloric acid (pH=1). The precipitate is removed by suction, washed several times with water and dried in vacuo.

Yield: 12.25 g (97% of the theoretical value) as a colourless solid

Elemental analysis: C 70.33 (70.47) H 6.25 (6.31) N 3.22 (3.18)

### e) 3-Benzyloxy-6-{[1,3-bis-(2-benzyloxy-1-benzyloxymethyl-ethoxy)-prop-2-yl]-carbamoyl}-4-nitrophenylcarbonyl-2-oxo-1,2-dihydropyridine-4-carboxylic acid

11.8 g (13.55 mmol) of the title compound from example 21d and 2.07 g (14.91 mmol) of 4-nitrophenol are dissolved in 150 ml of tetrahydrofuran. Then 3.08 g (14.91 mmol) of dicyclohexyl carbodiimide (Fluka) are added at 0° C. and stirred for 20 hrs at room temperature. The reaction mixture is filtered off, the filtrate concentrated until dry and the crude product recrystallised from diisopropyl ether.

Yield: 11.9 g (89% of the theoretical value) as a colourless solid

Elemental analysis: C 69.01 (69.38) H 5.79 (5.87) N 4.24 (4.03)

### f) 5-(bis-{2-[(3-Benzyloxy-6-{[1,3-bis-(2-benzyloxy-1-benzyloxymethyl-ethoxy)-prop-2-yl]-carbamoyl}-2-oxo-1,2-dihydropyridine-4-carbonyl)-amino]ethyl}amino)-4-[(3-benzyloxy-6-{[1,3-bis-(2-benzyloxy-1-benzyloxymethyl-ethoxy)-prop-2-yl]-carbamoyl}-2-oxo-1,2-dihydropyridine-4-carbonyl)-amino]-pentane carboxylic acid

685 mg (3.14 mmol) of 4-amino-5-[bis-(2-aminoethyl)-amino]-pentane carboxylic acid and 11.2 g (11.29 mmol) of the title compound from example 21e are dissolved in 50 ml of methylene chloride and stirred for three days at room temperature. The reaction mixture is extracted mit 30 ml of 1 N sodium hydroxide solution and with 30 ml of saturated sodium chloride solution, the organic phase dried with sodium sulfate, concentrated until dry and subjected to chromatography on silica gel (methylene chloride/methanol 20:1). The fractions containing the product are combined and concentrated.

Yield: 7.78 g (89% of the theoretical value) as a colourless solid

Elemental analysis: C 70.06 (70.34) H 6.46 (6.52) N 5.04 (4.99)

### g) 5-(bis-{2-[(3-Hydroxy-6-{[1,3-bis-(2-hydroxy-1-hydroxymethyl-ethoxy)-prop-2-yl]-carbamoyl}-2-oxo-1,2-dihydropyridine-4-carbonyl)-amino]ethyl}amino)-4-[(3-hydroxy-6-{[1,3-bis-(2-hydroxy-1-hydroxymethyl-ethoxy)-prop-2-yl]-carbamoyl}-2-oxo-1,2-dihydropyridine-4-carbonyl)-amino]-pentane carboxylic acid

1.5 g of palladium catalyst (10% Pd/C) are added to a solution of 7.0 g (2.52 mmol) of the title compound from example 21f in 100 ml of ethanol and hydrogenated for 48 hrs at room temperature. The mixture is filtered off the catalyst and the filtrate concentrated in vacuo until dry.

Yield: 3.61 g (quantitative) of a colourless solid

Elemental analysis: C 48.03 (48.42) H 6.22 (6.31) N 9.83 (9.71)

### h) Gadolinium complex of the 5-(Bis-{2-[(3-hydroxy-6-{[1,3-bis-(2-hydroxy-1-hydroxymethyl-ethoxy)-prop-2-yl]-carbamoyl}-2-oxo-1,2-dihydropyridine-4-carbonyl)-amino]ethyl}amino)-4-[(3-hydroxy-6-{[1,3-bis-(2-hydroxy-1-hydroxymethyl-ethoxy)-prop-2-yl]-carbamoyl}-2-oxo-1,2-dihydropyridine-4-carbonyl)-amino]-pentane carboxylic acid

3.0 g (2.10 mmol) of the title compound from example 21g are dissolved at reflux in 50 ml of tetrahydrofuran and 10 ml of methanol and 483 mg (2.10 mmol) of gadolinium trichloride hexahydrate dissolved in 5 ml of tetrahydrofuran/methanol (5:1) slowly added with heat resulting in the precipitation of a white precipitate. Then 1.5 ml of pyridine are added and heated at reflux for 18 hrs. After completion of the complexing, the mixture is concentrated in vacuo and subjected to chromatography on silica gel (solvent: dichloromethane/methanol/ammonia: 20/20/1). The fractions containing the product are combined and concentrated.

Yield: 2.33 g (67% of the theoretical value) of a powder with a slight greyish yellow tinge. Water content (Karl-Fischer): 4.5%

Elemental analysis (based on the anhydrous substance): C 43.34 (43.43) H 5.42 (5.44) Gd 9.95 (9.77) N 8.87 (8.91)

### Example 22

### a) 5-(bis-{2-[(3-Benzyloxy-1-{[1,3-bis-(2-benzyloxy-1-benzyloxymethyl-ethoxy)-prop-2-yl]-carbamoyl}-6-methyl-2-oxo-1,2-dihydropyridine-4-carbonyl)-amino]ethyl}-amino)-4-[(3-benzyloxy-1-{[1,3-bis-(2-benzyloxy-1-benzyloxymethyl-ethoxy)-prop-2-yl]-carbamoyl}-6-methyl-2-oxo-1,2-dihydropyridine-4-carbony)-amino]-pentane carboxylic acid

607 mg (2.78 mmol) of 4-amino-5-[bis-(2-aminoethyl)-amino]-pentane carboxylic acid and 9.63 g (10 mmol) of the title compound from example 20d are dissolved in 50 ml of methylene chloride and stirred for three days at room temperature. The reaction mixture is extracted with 30 ml of 1 N sodium hydroxide solution and with 30 ml of saturated sodium chloride solution, the organic phase dried with sodium sulfate, concentrated until dry and subjected to chromatography on silica gel (methylene chloride/methanol 20:1). The fractions containing the product are combined and concentrated.

Yield: 6.25 g (83% of the theoretical value) as a colourless solid

Elemental analysis: C 72.40 (72.66) H 6.82 (6.87) N 3.63 (3.59)

### b) 5-(bis-{2-[(3-Hydroxy-1-{[1,3-bis-(2-hydroxy-1-hydroxymethyl-ethoxy)-prop-2-yl]-carbamoyl}-6-methyl-2-oxo-1,2-dihydropyridine-4-carbonyl)-amino]ethyl}amino)-4-[(3-hydroxy-1-{[1,3-bis-(2-hydroxy-1-hydroxymethyl-ethoxy)-prop-2-yl]-carbamoyl}-6-methyl-2-oxo-1,2-dihydropyridine-4-carbony)-amino]-pentane carboxylic acid

1.5 g of palladium catalyst (10% Pd/C) is added to a solution of 6.0 g (2.52 mmol) of the title compound from example 22a in 100 ml of ethanol and hydrogenated for 48 hrs at room temperature. The mixture is filtered off the catalyst and the filtrate concentrated in vacuo until dry.

Yield: 2.23 g (quantitative) of a colourless solid.

Elemental analysis: C 51.51 (51.79) H 6.93 (6.99) N 7.25 (7.17)

### c) Gadolinium complex of the 5-(bis-{2-[(3-hydroxy-1-{[1,3-bis-(2-hydroxy-1-hydroxymethyl-ethoxy)-prop-2-yl]-carbamoyl}-6-methyl-2-oxo-1,2-dihydropyridine-4-carbonyl)-amino]ethyl}amino)-4-[(3-hydroxy-1-{[1,3-bis-(2-hydroxy-1-hydroxymethyl-ethoxy)-prop-2-yl]-carbamoyl}-6-methyl-2-oxo-1,2-dihydropyridine-4-carbony)-amino]-pentane carboxylic acid

2.0 g (1.48 mmol) of the title compound from example 22b are dissolved at reflux in 50 ml of tetrahydrofuran and 10 ml of methanol and 340 mg (1.48 mmol) of gadolinium trichloride hexahydrate dissolved in 5 ml of tetrahydrofuran/methanol (5:1) slowly added with heating, resulting in the precipitation of a white precipitate. 1.5 ml of pyridine are then added and heated at reflux for 18 hrs. After completion of the complexing, the mixture is concentrated in vacuo and subjected to chromatography on silica gel (solvent: dichloromethane/methanol/ammonia: 20/20/1). The fractions containing the product are combined and concentrated.

Yield: 1.72 g (73% of the theoretical value) of a powder with a greyish-yellow tinge Water content (Karl-Fischer): 5.1%

Elemental analysis (based on the anhydrous substance): C 46.24 (46.51) H 6.02 (6.09) Gd 10.44 (10.31) N 6.51 (6.47)

Gadolinium Complex Conjugates With Biomolecules (Examples 23-52)

The extremely favourable relaxivity properties of the novel inventive Gd complexes were demonstrated by preparing complex conjugates with biomolecules. It is thus possible to test the suitability of Gd complexes as biomolecule markers through immobilization with a macromolecule.

Examples 23-52 describe metal complex conjugates of the gadolinium complexes described above with biomolecules. The conjugates were prepared by the following general procedures I-V. The results are summarized in Table 1. In this “GP” stands for general procedure, “ACTH” for adrenocorticotropic hormon, “BSA” for bovine serum albumin, “HSA” for human serum albumin and “RP-18” denotes a reversed phase statioary chromotographic phase. The number of complexes per biomolecule was determined by ICP (inductively coupled plasma atomic emission spectroscopy).

General Procedure (GP) I: Albumin-amide Conjugates

3 mmol of the Gd complex acid are dissolved in 15 ml of DMF and, while cooling in ice, 380 mg (3.3 mmol) of N-hydroxysuccinimide and 681 mg of dicyclohexyl-carbodiimide are added, and preactivation is carried out while cooling in ice for 1 hour. The active ester mixture is added dropwise over the course of 30 minutes to a solution of 16.75 g (0.25 mmol) of albumin (human or bovine serum albumin) in 150 ml of phosphate buffer (pH 7.4) and stirred at room temperature for 2 hours. The batch solution is filtered, the filtrate is ultrafiltered through an AMICON® YM30 (cutoff 30 000 Da), the retentate is chromatographed on a Sephadex® G50 column, and the product fractions are freeze dried.

General Procedure (GP) II: Albumin-maleimide Conjugates

0.0438 mmol of the Gd complex maleimide in 1 ml of DMF is added to 0.84 g (0.0125 mmol) of bovine serum albumin (HSA), dissolved in 15 ml of phosphate buffer (pH 7.4), and stirred at room temperature for one hour. The batch solution is filtered, the filtrate is ultrafiltered through an AMICON® YM30 (cutoff 30 000 Da), the retentate is chromatographed on a Sephadex® G50 column, and the product fractions are freeze dried.

General Procedure (GP) III: Thiourea Conjugates

0.1 mmol of the Gd complex isothiocyanate in 5 ml of DMF is added to 0.84 g (0.0125 mmol) of bovine serum albumin (BSA), dissolved in 15 ml of phosphate buffer (pH 8.0), and stirred at room temperature for one hour. The batch solution is filtered, the filtrate is ultrafiltered through an AMICON® YM30 (cutoff 30 000 Da), the retentate is chromatographed on a Sephadex® G50 column, and the product fractions are freeze dried.

General Procedure (GP) IV: Amid Conjugates

3 mmol of the Gd complex acid are dissolved in 15 ml of DMF and, while cooling in ice, 380 mg (3.3 mmol) of N-hydroxysuccinimide and 681 mg of dicyclohexylcarbodiimide are added, and preactivation is carried out while cooling in ice for 1 hour. The active ester mixture is added dropwise to a solution of 2.5 mmol of amine component in 15-150 ml of DMF and stirred at room temperature over night. The batch solution is filtrated and chromatographed on silica gel.

General Procedure (GP) V: Maleimido-SH Conjugate

3 mmol of the Gd complex acid in 15 ml of DMF are added dropwise to 2.5 mmol SH component in 15-150 ml of DMF and stirred at room temperature for 1 hour. The batch solution is chromatographed on silicea gel.

TABLE 1

| | Educt | | | | number of | | | | |
|---|---|---|---|---|---|---|---|---|---|
| | Gd- | | | | complexes | | | Relaxivity* | Relaxivity* |
| | complex | | | | per | | | R1 (mM⁻¹ | R1 (mM⁻¹ |
| Example | example | | obtained | | biomolecule | | | s⁻¹) | s⁻¹) |
| No. | No. | conjugated with | from | GP | (ICP) | remarks | yield (%) | 20 MHz | 60 MHz |
| 23 | 1 | BSA | Sigma | I | 4.7 | — | quant. | 78.3 | 86.5 |
| 24 | 2 | BSA | Sigma | I | 5.1 | — | quant. | 77.2 | 86.1 |
| 25 | 3 | BSA | Sigma | I | 3.5 | — | quant. | 79.1 | 83.5 |
| 26 | 4 | BSA | Sigma | I | 3.9 | — | quant. | 69.3 | 78.2 |
| 27 | 5 | BSA | Sigma | I | 4.2 | — | quant. | 76.1 | 85.3 |
| 28 | 6 | BSA | Sigma | I | 6.5 | — | quant. | 76.9** | 85.6** |
| 29 | 7 | BSA | Sigma | II | 0.76 | — | quant. | 78.1 | 87.2 |
| 30 | 8 | BSA | Sigma | I | 4.8 | — | quant. | 77.2 | 88.1 |
| 31 | 9 | BSA | Sigma | III | 4.7 | — | quant. | 76.4 | 87.0 |
| 32 | 10 | BSA | Sigma | II | 0.83 | — | quant. | 78.2 | 86.9 |
| 33 | 11 | BSA | Sigma | I | 3.7 | — | quant. | 77.7 | 84.2 |
| 34 | 12 | BSA | Sigma | I | 0.78 | — | quant. | 79.0 | 88.2 |
| 35 | 13 | BSA | Sigma | I | 3.9 | — | quant. | 75.9 | 87.1 |
| 36 | 14 | BSA | Sigma | I | 4.6 | — | quant. | 67.8 | 78.3 |
| 37 | 15 | BSA | Sigma | I | 2.4 | — | quant. | 77.2 | 82.1 |
| 38 | 16 | HSA | Sigma | I | 5.9 | — | quant. | 71.3 | 80.9 |
| 39 | 17 | HSA | Sigma | I | 4.2 | — | quant. | 76.2 | 84.3 |
| 40 | 18 | HSA | Sigma | I | 4.8 | — | quant. | 64.3 | 76.2 |
| 41 | 19 | HSA | Sigma | I | 3.7 | — | quant. | 72.5 | 85.7 |
| 42 | 20 | HSA | Sigma | I | 3.1 | — | quant. | 70.1 | 81.0 |
| 43 | 21 | HSA | Sigma | I | 5.2 | — | quant. | 75.3 | 86.1 |
| 44 | 22 | HSA | Sigma | I | 4.8 | — | quant. | 78.6 | 83.2 |
| 45 | 16 | ACTH (1-17) | BACHEM | IV | 1.0 | purification on | 74 | n.d. | n.d. |
| | | | | | | RP-18 | | | |
| 46 | 17 | H-β-Ala-Phe | BACHEM | IV | 1.0 | purification on | 94 | n.d. | n.d. |
| | | | | | | RP-18 | | | |
| 47 | 18 | H-Arg-Gly-Asp- | BACHEM | V | 1.0 | purification on | 92 | n.d. | n.d. |
| | | Cys-OH | | | | RP-18 | | | |
| 48 | 19 | H-Asp-Leu-Trp- | BACHEM | IV | 2.0 | purification on | 88 | n.d. | n.d. |
| | | Gln-Lys-OH | | | | RP-18 | | | |
| 49 | 18 | H-Ala-His-Lys- | BACHEM | IV | 2.0 | purification on | 96 | n.d. | n.d. |
| | | OH | | | | RP-18 | | | |
| 50 | 10 | Homo- | BACHEM | V | 1.0 | — | 72 | n.d. | n.d. |
| | | glutathione | | | | | | | |
| 51 | 10 | HSA | Sigma | V | 0.79 | — | quant. | 78.3 | 83.9 |
| 52 | 10 | Guanyl-Cys-OH | BACHEM | V | 1.0 | — | 87 | n.d. | n.d. |

*calculated in water (30° C.);
**measured in water (30° C.)

### Example 53

In this example, the relaxivity of a prior art substance was measured for comparison purposes. The comparative substance employed was a conjugate of the following formula (1:1 complex):

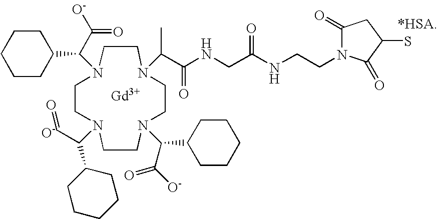

The measurement took place in aqueous solution at +30° C. T1 relaxivities of 40.6 at 20 MHz and 21.7 at 60 MHz were measured.

By contrast, the T1 relaxivities of the inventive conjugates at 60 MHz are higher than at 20 MHz, as can be seen in Table 1 above. The inventive conjugates thus have a higher relaxivity at higher field, so that they are particularly suitable for use together with clinical NMR diagnostic instruments.

1. A conjugate of the general formula I:
(K)₃—A—U—X′-Bio I,
in which K is independently of one another a radical: 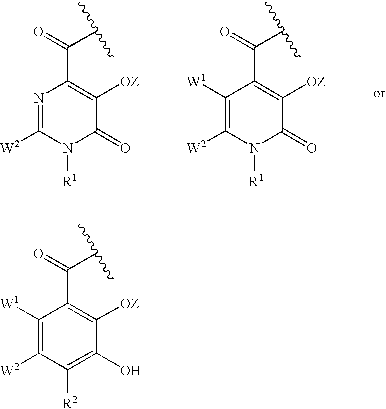
in which Z is a hydrogen atom or a metal ion equivalent,
R¹ is a hydrogen atom or a straight-chain or branched, saturated or unsaturated C₁₋₁₀-alkyl radical which is optionally interrupted by 1-3 oxygen atoms, 1-3 nitrogen atoms and/or 1-3 —NR³ radicals, is optionally substituted by 1-4 hydroxy groups, 1-2 carboxyl (optionally present in protected form), 1-2 —SO₃H (optionally present in protected form), 1-2 —PO₃H₂ groups and/or 1-2 halogen atoms, and/or in which optionally 1-2 carbon atoms are present as carbonyl groups, where the alkyl radical or a part of the alkyl radical may be in cyclic form,
R² is a hydrogen atom, a straight-chain or branched, saturated or unsaturated C₁₋₁₀-alkyl radical which is optionally interrupted by 1-3 oxygen atoms, 1-3 nitrogen atoms and/or 1-3 —NR³ radicals, is optionally substituted by 1-2 hydroxy groups, 1-2 carboxyl, 1-2 —SO₃H, 1-2 —PO₃H₂ groups and/or 1-2 halogen atoms, and/or in which optionally 1-2 carbon atoms are present as carbonyl groups, where the alkyl radical or a part of the alkyl radical may be in cyclic form, —COOH—, halogen, —CONR³R⁴, —SO₃H or —PO₃H₂,
R³ and R⁴ are independently of one another a hydrogen atom or a straight-chain, branched or cyclic, saturated or unsaturated C₁₋₁₀-alkyl radical which is optionally substituted by 1-4 hydroxy groups or interrupted by 1-2 oxygen atoms,
W¹ and W² are independently of one another a radical R¹ or —CONR³R⁴.
A is a radical: 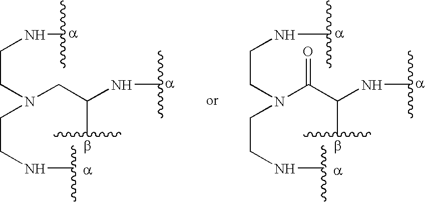
in which the positions α are linked to K and the positions β are linked to U,
U is a direct linkage or a straight-chain or branched, saturated or unsaturated C₁₋₂₀-alkylene radical which is optionally interrupted by 1-4 oxygen atoms, 1-4 sulphur atoms, 1-4 nitrogen atoms, 1-4 —NR³ radicals, 1-4 —NHCO radicals, 1-4 —CONH radicals, 1-4 —O—P(═O)(OH)—O— radicals and/or 1-2 arylene radicals, is optionally substituted by 1-3 straight-chain, branched or cyclic, saturated or unsaturated C₁₋₁₀-alkyl radicals, 1-3 hydroxy groups, 1-3 carboxyl groups, 1-3 aryl groups, 1-3 halogen atoms and/or 1-3 —O—C₁₋₆-alkyl groups (where the alkyl radical is straight-chain, branched or cyclic, saturated or unsaturated), and/or in which optionally 1-3 carbon atoms may be present as carbonyl groups, where the alkylene radical or a part of the alkylene radical may be in cyclic form, and
X is a group able to enter into a reaction with a biomolecule, and
Bio is the residue of a biomolecule and the salts thereof.

2. A conjugate according to claim 1, in which U is selected from the group consisting of —CH₂—CH₂—, —CH₂—CH₂—CO—NH—CH₂—CH₂—, —CH₂—CO—NH—CH₂—, —CH (CH₃) —CO—NH—CH₂—CO—NH—CH₂—CH₂—, —CH₂-phenylene-, -phenylene-, -cyclohexylene-, —CH₂-phenylene-O—CH₂—, —CH₂-phenylene-O—CH₂—CO—NH—CH₂—CH₂—, -phenylene-O—CH₂—, —CO—phenylene-, —CO-phenylene-CO—NH—CH₂—CH₂—, —(CH₂)₄—, —(CH₂)₄—NH—CO—CH₂—CH₂— and —(CH₂)₄—NH—CO—CH₂—O—CH₂—, where these radicals are linked in the direction of reading on the left to A and in the direction of reading on the right to X.

3. A conjugate according to claim 1 or 2, in which X′ is a radical of a group X and X is selected from the group consisting of carboxyl, activated carboxyl, amino, isocyanate, isothiocyanate, hydrazine, semicarbazide, thiosemicarbazide, chloroacetamide, bromoacetamide, iodoacetamide, acylamino, mixed anhydrides, azide, hydroxide, sulphonyl chloride, carbodiimide, pyridyl-CH═CH₂ and radicals of the formulae: 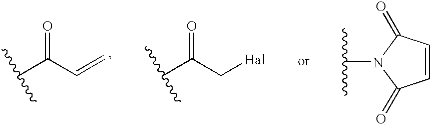
in which Hal is a halogen atom.

4. A conjugate according to claim 3, in which the activated carboxyl group is selected from:

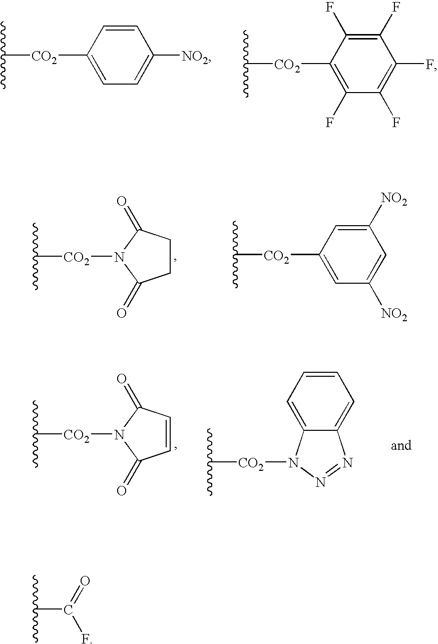

5. A conjugate according to claim 1, in which the biomolecule is selected from the group consisting of biopolymers, proteins, synthetically modified biopolymers, carbohydrates, antibodies, DNA and RNA fragments, β-amino acids, vector amines for importation into the cell, biogenic amines, pharmaceuticals, oncological preparations, synthetic polymers directed at a biological target, steroids, prostaglandins, Taxol and its derivatives, endothelins, alkaloids, folic acid and its derivatives, bioactive lipids, fats, fatty acid esters, synthetically modified mono-, di- and triglycerides, liposomes which are derivatized on the surface, micelles of natural fatty acids or of perfluoroalkyl compounds, porphyrins, texaphrins, extended porphyrins, cytochromes, inhibitors, neuraminidases, neuropeptides, immunomodulators, endoglycosidases, substrates which are attacked by the enzymes, calmodulin kinase, casein kinase II, glutathione S-transferase, heparinase, matrix metalloproteases, β-insulin receptor kinase, UDP-galactose 4-epimerase, fucosidases, G-proteins, galactosidases, glycosidases, glycosyl transferases and xylosidase, antibiotics, vitamins and vitamin analogues, hormones, DNA intercalators, nucleosides, nucleotides, lectins, vitamin B12, Lewis-X and related substances, psoralens, diene/triene antibiotics, carbacyclins, VEGF, somatostatin and its derivatives, biotin derivatives, antihormones, tumour-specific proteins and synthetics, polymers which accumulate in acidic or basic regions of the body, myoglobins, apomyoglobins, neurotransmitter peptides, tumour necrosis factors, peptides which accumulate in inflamed tissues, blood pool reagents, anions and cation transporter proteins, polyesters, polyamides and polyphosphates.

6. A conjugate according to claim 1, in which at least two of the radicals Z are a metal ion equivalent of a paramagnetic element of atomic numbers 21-29, 42, 44 or 58-70.

7. A process for preparing a conjugate of the general formula I:
(K)₃—A—U—X′-Bio I,
in which K, A, U, X′ and Bio are as defined in claim 1, in which a compound of the general formula II:
(K)₃—A—U—X II,
in which K, A and U are as defined above and X is a group which is able to enter into a reaction with a biomolecule, is reacted with a biomolecule and if desired is subsequently reacted in a manner known per se with at least one metal oxide or metal salt of a desired element, and where appropriate subsequently acidic hydrogen atoms still present in the complexes obtained in this way are replaced wholly or partly by cations of inorganic and/or organic bases, amino acids or amino amides.

8. A pharmaceutical composition comprising at least one physiologically acceptable conjugate according to claim 6, if desired with the additives customary in pharmaceuticals.

9. Use of a conjugate according to claim 6 for preparing agents for NMR diagnosis.

10. A conjugate according to claim 1 or 2, in which the activated carboxyl group is selected from:

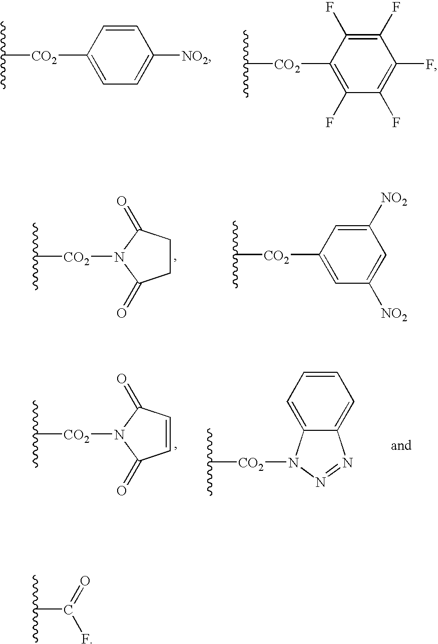

11. A conjugate according to claim 1 or 2, in which the biomolecule is selected from the group consisting of biopolymers, proteins, synthetically modified biopolymers, carbohydrates, antibodies, DNA and RNA fragments, β-amino acids, vector amines for importation into the cell, biogenic amines, pharmaceuticals, oncological preparations, synthetic polymers directed at a biological target, steroids, prostaglandins, Taxol and its derivatives, endothelins, alkaloids, folic acid and its derivatives, bioactive lipids, fats, fatty acid esters, synthetically modified mono-, di- and triglycerides, liposomes which are derivatized on the surface, micelles of natural fatty acids or of perfluoroalkyl compounds, porphyrins, texaphrins, extended porphyrins, cytochromes, inhibitors, neuraminidases, neuropeptides, immunomodulators, endoglycosidases, substrates which are attacked by the enzymes, calmodulin kinase, casein kinase II, glutathione S-transferase, heparinase, matrix metalloproteases, β-insulin receptor kinase, UDP-galactose 4-epimerase, fucosidases, G-proteins, galactosidases, glycosidases, glycosyl transferases and xylosidase, antibiotics, vitamins and vitamin analogues, hormones, DNA intercalators, nucleosides, nucleotides, lectins, vitamin B12, Lewis-X and related substances, psoralens, diene/triene antibiotics, carbacyclins, VEGF, somatostatin and its derivatives, biotin derivatives, antihormones, tumour-specific proteins and synthetics, polymers which accumulate in acidic or basic regions of the body, myoglobins, apomyoglobins, neurotransmitter peptides, tumour necrosis factors, peptides which accumulate in inflamed tissues, blood pool reagents, anions and cation transporter proteins, polyesters, polyamides and polyphosphates.

12. A conjugate according to claim 1 or 2, in which at least two of the radicals Z are a metal ion equivalent of a paramagnetic element of atomic numbers 21-29, 42, 44 or 58-70.

13. A process for preparing a conjugate of the general formula I:
(K)₃—A—U—X′-Bio I,
in which K, A, U, X′ and Bio are as defined in claim 1, in which a compound of the general formula II:
(K)₃—A—U—X II,
in which K, A and U are as defined above and X is a group which is able to enter into a reaction with a biomolecule, is reacted with a biomolecule to form a complex.

14. A process according to claim 13, further comprising reacting said complex with at least one metal oxide or metal salt of a desired element.

15. A process according to claim 14, further comprising replacing wholly or partly one or more acidic hydrogen atoms still present in the complex with one or more cations of inorganic and/or organic bases, amino acids or amino amides.

16. A pharmaceutical composition comprising at least one physiologically acceptable conjugate according to according to any of claims 1, 2, 5 or 6, if desired with the additives customary in pharmaceuticals.
